(19) United States
(12) Patent Application Publication
(10) Pub. No.: US 2019/0299115 A1
(43) Pub. Date: Oct. 3, 2019

(54) SOLVENT-FREE METHODS AND SYSTEMS FOR EXTRACTION OF PHYTOCHEMICALS FROM PLANTS INCLUDING PLANTS OF THE CANNABACEAE

(71) Applicant: Frank A. LOPA, Staten Island, NY (US)

(72) Inventor: Frank A. LOPA, Staten Island, NY (US)

(21) Appl. No.: 16/199,051

(22) Filed: Nov. 23, 2018

Related U.S. Application Data
(63) Continuation-in-part of application No. 15/669,907, filed on Aug. 5, 2017, now Pat. No. 10,272,360.
(60) Provisional application No. 62/770,665, filed on Nov. 21, 2018.

(51) Int. Cl. B01D 3/10; C11B 1/10; C11B 1/12; B01D 3/38; B01D 7/00; A01H 6/28

(52) U.S. Cl. CPC B01D 3/101; C11B 1/10; A01H 6/28; B01D 3/38; B01D 7/00; C11B 1/12

(57) ABSTRACT

A method and system of and for extraction or removal of phytochemicals from plants, including those of the plant family Cannabaceae. More specifically, a method and system for extracting essential oils from plants, such as cannabis, without the use of a solvent.

### CROSS-REFERENCE TO RELATED APPLICATIONS

The present application is a continuation-in-part of and claims the benefit of priority to U.S. application Ser. No. 15/669,907, entitled Phytochemical Extraction System and Methods to Extract Phytochemicals from Plants Including Plants of the Family Cannabaceae Sensu Stricto, filed on Aug. 5, 2017. The entirety of said application, its Figures, and its Appendices, are all hereby incorporated by reference.

### FIELD OF THE INVENTION

The present invention is directed to advanced extraction systems and methods to extract or remove phytochemicals from plants, including those of the plant family Cannabaceae. The present invention is directed to plant oil extraction and, more specifically, the use of a method and system for extracting essential oils from plants, such as cannabis, without the use of a solvent.

This application describes various advances and improvements to U.S. patent application Ser. No. 15/669,907, mentioned above, and also draws upon information disclosed in U.S. Provisional Patent Application No. 62/770,665, entitled Systems and Methods to Extract Phytochemicals from Plants Including Plants of the Cannabaceae, filed on Nov. 21, 2018. The entirety of said provisional patent application, its Figures, its Appendices, all other parts thereof, are all hereby incorporated by reference.

### BACKGROUND OF THE INVENTION

Many plants, such as plants from the family Cannabaceae, have many medicinal and therapeutic activity and uses. The medicinal and therapeutic activity of plants is primarily due to the active biological compounds or phytochemicals that the plants contain. The activity of phytochemicals is typically associated to a specific plant species of which a combination of compounds act in concert or harmony to result in a healing or medicinal outcome. Regardless of the concentration in a phyto-biomass, it is desirable to extract specific compounds, or produce an enriched and/or purified extract from plants, which can be then used for medicinal and pharmaceutical formulations.

Known extraction methods and systems which have been used to separate phytochemicals from plants, and produce enriched compounds, include maceration, decoction, distillation, sublimation, and extraction with aqueous and non-aqueous solvents.

Maceration may be defined as the extraction of a compound in a solvent at ambient room temperature with daily shaking or stirring. After a selected period, the solid material is separated from the solution. Variation on the method includes agitation of the macerate and the use of temperatures up to approximately 50° C. A variation of the method includes preparation of tinctures and extracts from low-density plant material using various strengths of ethanol as a solvent.

Decoction has been used since antiquity for the preparation of medicines; and customarily in traditional Chinese medicine. The quantity of herbs required for one day's treatment are placed into a vessel and hot or boiling water is added. The vessel may be brought to a boil and allowed to simmer for one or more hours. Once cooled, solid particles are filtered out and the decoction administered orally.

Maceration and decoction rely on short path diffusion, where inactive constituents such as lecithins, flavonoids, glycosides and sugars act to solubilize compounds which, in a pure state, are soluble in the solvent. A disadvantage of maceration and decoction with water or low concentrations of ethanol is that a large quantity of inert material typically having no therapeutic value must be removed. This inert material may consist of plant cell elements including, but not limited to fats, waxes, carbohydrates, proteins, and sugars. This may contribute to microbiological spoilage of a resulting product if not used promptly or further refined or preserved in some fashion. If dried, such extracts tend to be hygroscopic and difficult to formulate. The inert material may also affect how active phyto-elements are absorbed in and from a finished formulation.

Maceration and decoction are still widely used in situations where convenience outweighs precise dosage accuracy. Macerate and/or percolate solvents may be removed by evaporation at temperatures below 100° C. provided the correct solvent is used.

A wide range of processes based on the use of non-aqueous solvents to extract compounds from plants are known and taught in the prior art. Solvents employed may be miscible or immiscible with water and vary in efficacy. Techniques used to extract compounds from plants include liquid and solid extraction, liquid and gas chromatography and other separation and fractioning techniques.

Traditionally, for plant materials, ethyl alcohol in various concentrations is used to extract active substances. Tinctures are ethanol solutions easily produced and well described in most major pharmacopoeias. Where the final concentration of alcohol is greater than approximately 20% by volume, the tincture remains microbiologically stable and widely used in compounding prescriptions. Ethanol extract substances such as glycosides, flavonoids and alkaloid salts are examples of compounds known to be biologically active. Ethanol also extracts considerable amounts of plant pigment, such as chlorophyll and carotenoids. By using higher alcohol strengths, lipid-soluble material may be extracted. Tinctures typically contain less inert material than macerates or decoctions, but are still complex mixtures of plant chemical elements. Where alcohol is not required or desired, a tincture may be evaporated to produce ethanol free extracts.

Lipid solvents are also used to extract lipid soluble chemical elements from a phyto-biomass. Examples are chlorinated solvents such as dichloromethane, chloroform, carbon-tetrachloride, hexane, ether, fluorinated hydrocarbons, and supercritical fluid extraction with inert agents such as carbon dioxide.

Using chlorinated solvents is highly disadvantageous for phyto-biomass extraction because of extreme toxicity; and because for medicinal or pharmaceutical use such toxic solvents must be removed by various means before administration. Hexane and other petroleum-based solvents have good solvent activity; however, they must also be completely removed from any end product, and also carry the risk of fire and explosion during use.

Distillation and sublimation have been widely used to separate components of phyto-chemicals which have boiling points close to water (100° C.) at sea-level atmospheric pressure (14 psi). Chemical separation by distillation is widely used in the preparation of essential oils and also petrochemicals.

Known methods and systems used to extract compounds include: U.S. Pat. Nos. 1,679,728, 2,198,412, 2,414,418, 3,270,437, 3,936,489, 4,279,824, 5,372,680, 5,516,923, 5,525,746, 6,350,351, 6,365,416, 6,403,126, 6,730,519, 6,946,150, 7,025,992, 7,291,250, 7,344,736, 7,524,881, 7,592,468, 7,622,140, 7,700,368, 8,343,553, 8,445,034, 8,530,679, 8,673,368, 8,846,409, 8,859,793, 8,895,078, 8,906,956, 9,022,040, 9,034,395, 9,034,395, 9,035,130, 9,044,390, 9,186,386, 9,199,960, 9,205,063, 9,327,210, 9,333,441, 9,358,259, 9,592,457, 9,649,349, 9,649,349, 9,649,575, 9,655,936, 9,655,937, 9,669,326, 9,669,328, US20020039795, US20020086438, US20030017216, US20030050334, US 20040033280, US20040049059, US20040147767, US20040147769, US20050049298, US 20060167283, US20080031977, US20080167483, US20100119606, US20100168448, US20110100894, US20110133120, US20110201836, US20110256245, US20120263804, US20130079531, US20130149322, US20130256245, US20120263804, US20130079531, US20130149322, US20130251824, US20140113010, US20140114084, US20140248379, US20140341934, US20150105569, US20150119592, US20150203434, US20150258153, US20150297653, US20150297654, US20150375136, US20160038437, US20160074450, US20160074451, US20160106705, US20160136541, US20160201009, US20160136541, US20160201009, US20160213720, US20160228787, US20160279183, US20160324909, US20160326130, US20160346339, US20160360721, US20170008870, US20170020943, US 20170020944, US20170049830, US20170051231, US20170106030, US20170119040.

It is desired to provide a method and system to overcome the above-mentioned and other disadvantages in the prior art by and for removing phytochemicals and plant compounds from plant material or a phytochemical composition without adversely or undesirably by heat affecting the extracted phytochemicals themselves.

It is desired to provide a method and system that overcomes the above-mentioned and other disadvantages in the prior art and by and for removing phytochemicals from plant material or phytochemical compositions from the plant family Cannabaceae without adversely or undesirably by heat affecting the extracted phytochemicals themselves.

### SUMMARY OF THE INVENTION

It is an object of the present invention to provide a method and system of and for extracting or separating a phytochemical from plant material or a phytochemical composition.

It is an object of the present invention to provide a method and system of and for extracting or separating a phytochemical from plant material or a phytochemical composition without utilizing a solvent.

It is an object of the present invention to provide a method and system of and for extracting or separating a phytochemical from plant material or a phytochemical composition across a range of temperatures (e.g., ca. 15° C. to ca. 230° C.) and vacuum qualities (e.g., low, medium, and high vacuum (e.g., below ca.760 Torr to ca.1×10⁻⁶ Torr)).

These and other objects of the invention are achieved by providing a system to extract a phytochemical from plant material or a phytochemical composition comprising: a vacuum chamber configured to hold plant material or a phytochemical composition and to maintain a vacuum; an evacuation pump configured to create a vacuum within the vacuum chamber; and a collection chamber in fluid communication with the vacuum chamber; wherein when plant material or the phytochemical composition is placed in the vacuum chamber, the amount of vacuum created in the vacuum chamber by and with the evacuation pump is sufficient to cause at least one phytochemical to volatizes and/or precipitate from the plant material or the phytochemical composition and collect in the collection chamber without using a solvent, thereby creating a solvent-less phytochemical extract.

In certain embodiments of the inventive system, at least one valve is included within the system to facilitate return of the vacuum chamber to ambient atmospheric pressure.

In certain embodiments of the inventive system, upon actuation of the at least one valve the collection chamber is recompressed.

In certain embodiments of the inventive system, the at least one valve enables an explosive return of the vacuum chamber to ambient atmospheric pressure.

In certain embodiments of the inventive system, the at least one valve enables explosive recompression of the plant material within the collection chamber.

In certain embodiments of the inventive system, a pressurized gas or air reservoir in fluid communication with the at least one valve is included, wherein upon actuation of the at least one valve the vacuum chamber is compressed to approximately the pressure of the pressurized gas or air reservoir.

In certain embodiments of the inventive system, a second evacuation pump in fluid communication with the collection chamber is included capable of evacuating the collection chamber when the evacuation pump in fluid communication with the vacuum chamber is actuated and creating at least a partial vacuum in the vacuum chamber.

In certain embodiments of the inventive system, a filter or trap is included wherein upon return of the vacuum chamber to ambient atmospheric pressure, the at least one phytochemical is collected in or with the filter or trap.

In certain embodiments of the inventive system, the plant material is from and belongs to the plant family Cannabaceae.

In certain embodiments of the inventive system, the phytochemical composition includes a cannabinoid.

In certain embodiments of the inventive system, the heat source comprises combustion of a fuel.

In certain embodiments of the inventive system, the heat source comprises an electrical heat element. The electric heat element can be conductive (resistive) heating, IR heating (e.g., direct and indirect heating of the plant material or phytochemical composition via IR radiation), and laser-based heat source.

In certain embodiments of the inventive system, the heat source comprises a heated gas.

In certain embodiments of the inventive system, the heated gas is air.

In certain embodiments of the inventive system, the collected phytochemical includes a cannabinoid.

In certain embodiments of the inventive system, the solvent-less phytochemical extract includes one or more of active cannabinoids, inactive cannabinoids, terpenes, monoterpenes, alkaloids, flavonoids, and/or combinations thereof.

In certain embodiments of the inventive system, the collection chamber is located within or is part of the vacuum chamber.

In certain embodiments of the inventive system, the phytochemical collects in the collection chamber without using a solvent, thereby creating a solvent-less phytochemical extract.

In certain embodiments of the inventive system, the collection chamber and/or the trap or filter is cooled to a temperature below the temperature of the vacuum chamber to more effectively and efficiently collect the at least one phytochemical.

In certain embodiments of the inventive system, the system includes a dispenser chamber that is arranged to “feed” plant material or a phytochemical composition to or within the vacuum chamber. The dispenser may be within or outside the vacuum chamber. The dispenser may be in fluid communication with a holder located inside the vacuum chamber, thereby depositing plant material or a phytochemical composition onto (or into) the holder.

In certain embodiments of the inventive system, the system includes a stirrer that is arranged to stir plant material or a phytochemical composition inside the vacuum chamber. The stirrer may be arranged on or near the holder for stirring said material/composition on or within the holder.

In certain embodiments the holder is cylindrical with one side arranged to accept a material/composition from a dispenser and another side arranged to dispense with the ABV (“already been vaped”) material/composition via an opposing side of the cylinder. A holder may alternatively be other non-cylindrical hollow shapes such as a (substantially) sphere, cuboid, cone, cube, hexagonal prism, etc. The holder may be rotatably connected on one or more sides to allow rotation of the holder for “tumbling” the material/composition held therein. The holder may be arranged at an angle for gravity-assisted disposal of the material/composition. The holder may have ridges, bumps, or other features arranged on the inside of the holder. The holder may be operably coupled to one or more vibration elements for vibrating a material or composition within the holder.

In some embodiments, the heat source may surround at least a portion of the heat source for vaporization the material/composition. In some embodiments, the heat source may be an IR heat source that at least substantially surrounds a cylindrical holder. In some embodiments, the cylinder may be a transparent or semi-transparent material such as quartz, which is substantially “invisible” or transparent to the heat source's emitted wavelength to allow for an efficient transfer of the emitted energy to the material/composition (e.g., transparent across a portion of the EM spectrum).

In certain embodiments of the inventive system, at least one processor, at least one memory, at least one software program, and at least one configurable hardware device in wired or wireless communication with at least one temperature sensor, at least one pressure and/or vacuum sensor, at least one valve control solenoid, and at least one temperature control solenoid is included to provide digital command and control of the system.

Objects of the invention are achieved by providing a method of and for extracting a phytochemical from plant material or a phytochemical composition, the method comprising the steps of: providing a vacuum chamber configured to hold plant material or a phytochemical composition and maintain a vacuum; providing an evacuation pump configured to create a vacuum within the vacuum chamber, providing a collection chamber in fluid communication with the vacuum chamber; wherein when plant material or a phytochemical composition is placed in the vacuum chamber, the amount of vacuum created in the vacuum chamber by and with the evacuation pump sufficient to cause at least one phytochemical to volatizes and/or precipitate from the plant material or phytochemical composition and collect in the collection chamber without using a solvent, thereby creating a solvent-less phytochemical extract.

In certain embodiments, the inventive method includes the further step of providing a heat source, wherein the heat source increases the temperature within the vacuum chamber, the vacuum temperature is below 100° C., and the temperature causes the volatilization of the at least one phytochemical.

In certain embodiments, the inventive method includes the further step of providing at least one valve within the system to facilitate return of the vacuum chamber to ambient atmospheric pressure.

In certain embodiments, the inventive method includes the further step of providing at least one valve enabling an explosive return of the vacuum chamber to ambient atmospheric pressure.

In certain embodiments, the inventive method includes the step of providing at least one valve enabling explosive recompression of the chamber.

In certain embodiments, the inventive method includes the further step of providing a pressurized gas or air reservoir in fluid communication with the at least one valve, wherein upon actuation of the at least one valve the vacuum chamber is compressed to approximately the pressure of the pressurized gas or air reservoir.

In certain embodiments, the inventive method includes the further step of providing a second evacuation pump in fluid communication with the collection chamber capable of evacuating the collection chamber when the evacuation pump in fluid communication with the vacuum chamber is actuated and thereby creating at least a partial vacuum in the vacuum chamber.

In certain embodiments, the inventive method includes the further step of providing a filter or trap wherein upon return of the vacuum chamber to ambient atmospheric pressure, the at least one phytochemical is collected in or with the filter or trap.

In certain embodiments of the inventive method, the plant material is from and belongs to the plant family Cannabaceae.

In certain embodiments of the inventive method, the phytochemical composition includes a cannabinoid.

In certain embodiments of the inventive method, the solvent-less phytochemical extract includes at least one of cannabinoids, terpenes, and combinations thereof.

In certain embodiments of the inventive method, the heat source comprises an electrical heating element.

In certain embodiments of the inventive method further comprising cooling the collection chamber and/or the trap or filter to a temperature below the temperature of the vacuum chamber to more effectively and efficiently collect the at least one phytochemical.

In certain embodiments of the inventive method, at least one processor, at least one memory, at least one software program, and at least one configurable hardware device in wired or wireless communication with at least one temperature sensor, at least one pressure and/or vacuum sensor, at least one valve control solenoid, and at least one temperature control solenoid is provided to enable digital command and control of the system.

Objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 is a schematic diagram depicting an embodiment of the inventive method and system;

FIG. 2 is a schematic diagram depicting an embodiment of the inventive method and system;

FIGS. 3A-3B are schematic diagrams depicting embodiments of the inventive method and system;

FIGS. 4A-4H are schematic diagrams depicting embodiments of the inventive method and system;

FIGS. 5A-5C are schematic diagrams depicting embodiments of the inventive method and system;

FIGS. 6A-6C are schematic diagrams depicting embodiments of the inventive method and system;

FIG. 7 is a side, partially cut-away view of an embodiment of the inventive system;

FIG. 8 is a side, partially cut-away view of an embodiment of the inventive system;

FIG. 9 is an isometric view of an embodiment of the inventive system;

FIGS. 10A and 10B show various views of an embodiment of the inventive system;

FIGS. 11A and 11B are respectively cross-sectional and top view of embodiments of the inventive system;

FIG. 12 is a cross-sectional view of embodiments of the inventive system;

FIGS. 13A and 13B are cross-sectional views of embodiments of the inventive system;

FIG. 14 is a cross-sectional view of embodiments of the inventive system;

FIG. 15 is a side, partially cut-away view of an embodiment of the inventive system;

FIG. 16 is a side, partially cut-away view of an embodiment of the inventive system;

FIG. 17 is a schematic diagram depicting an embodiment of the inventive method and system;

FIG. 18 is a schematic diagram depicting an embodiment of the inventive method and system;

FIG. 19 is a side, partially cut-away view of an embodiment of the inventive system;

FIGS. 20A-C is a side, partially cut-away view of an embodiment of the inventive system;

FIG. 20D is a side view of an embodiment of the inventive system;

FIGS. 20E and 20F are cross-sectional view of an embodiment of the inventive system;

FIGS. 21 and 22 are cross-sectional views of embodiments of the inventive system;

FIG. 23 is a cross-sectional, partially schematic view of an embodiment of the inventive system;

FIG. 24 is a schematic diagram depicting an embodiment of the inventive method and system;

FIG. 25 is a side, partially cut-away view of an embodiment of the inventive system;

FIG. 26 is a schematic diagram depicting an embodiment of the inventive method and system;

FIGS. 27A-C show embodiments of the inventive method and system;

FIG. 28 depicts prior-art extraction processes with which the inventive method and system are contrasted; and

FIG. 29 depicts lab test results described in Example 1.

### DETAILED DESCRIPTION OF THE INVENTION

In the following description, numerous details are set forth for the purpose of example and explanation; however, one of ordinary skill in the art will realize that the invention may be practiced without the use of these specific details.

While not wishing to be bound by any one theory or combination of theories, it is believed and proven by instant inventor experimentation that utilizing at least a partial vacuum to reduce the temperature at which a phytochemical volatizes and/or precipitates from plant material or a phytochemical composition may be used to collect one or more phytochemicals, which can be used for medical, industrial, and scientific purposes.

While not wishing to be bound by any one theory or combination of theories, it is believed and proven by instant inventor experimentation that utilizing at least a partial vacuum to reduce the temperature at which a phytochemical volatizes and/or precipitates from plant material or a phytochemical composition may be used to collect one or more phytochemical, without causing substantial undesired alteration and/or degradation of the extracted phytochemical, which can be used for medical, industrial, and scientific purposes.

The figures and description below provide numerous embodiments and exemplary configurations whereby one of ordinary skill in the art can utilize at least a partial vacuum to reduce the temperature at which a phytochemical volatizes and/or precipitates from plant material or a phytochemical composition to collect one or more phytochemicals. Additional embodiments and configurations will be readily apparent to those of skill in the art upon reading the instant disclosure.

As used herein, “substantially maintain vacuum” shall mean retaining the same or similar quality of vacuum, even if there's some fluctuation to an established vacuum value. For example, maintaining one of a low, medium, and high vacuum quality or only switching “one level” of vacuum quality (e.g., High to Medium or Medium to Low):

| Vacuum quality | Torr |
|---|---|
| Atmospheric pressure | 760 |
| Low vacuum | 760 to 25 |
| Medium vacuum | 25 to 1 × 10⁻³ |
| High vacuum | 1 × 10⁻³ to 1 × 10⁻⁹ |

Providing plant material or a phytochemical composition “onto or into” the holder envisions embodiments where a holder may be substantially planar (thus the material/composition is “on” such holders) or defines a cavity (thus the material/composition is “within” or “in” such holders).

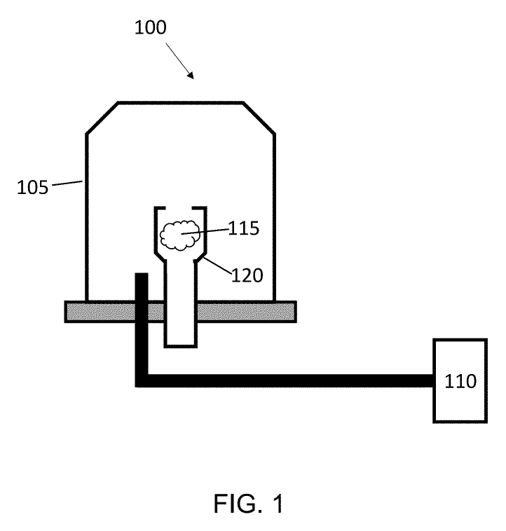

As depicted in FIG. 1, in an embodiment of the inventive method and system (100), a vacuum chamber (105) configured for and capable of maintaining at least a partial vacuum is in fluid communication via a conduit with an evacuation pump (110) (e.g., a turbomolecular pump or a rough pump). Plant material or a phytochemical composition (115) is placed in the vacuum chamber (105) and the evacuation pump (110) actuated to produce at least a partial vacuum in the vacuum chamber (105) adequate to cause volatization and/or precipitation of at least one phytochemical for collection. In some embodiments, the pressure of the partial vacuum adequate to cause volatization and/or precipitation of at least one phytochemical for collection is about 760 to 25 Torr. In some embodiments, the pressure of the partial vacuum adequate to cause volatization and/or precipitation of at least one phytochemical for collection is about 25 to 1×10⁻³ Torr. In some embodiments, the pressure of the partial vacuum adequate to cause volatization and/or precipitation of at least one phytochemical for collection is about 1×10⁻³ to 1×10⁹ Torr. In some embodiments, the pressure of the partial vacuum adequate to cause volatization and/or precipitation of at least one phytochemical for collection is in the millitor range. In some embodiments, the pressure of the partial vacuum adequate to cause volatization and/or precipitation of at least one phytochemical for collection is in the sub millitor range. In some embodiments, the pressure of the partial vacuum adequate to cause volatization and/or precipitation of at least one phytochemical for collection is about 1×10⁻⁴ Torr or about 1 to 1×10⁻⁵ Torr or about 1×10⁻⁶ Torr or about 1×10⁻⁷ Torr about a 1×10⁻⁸ Torr.

It is contemplated that the plant material or phytochemical composition (115) may be placed and held in the vacuum chamber (105) by many and varied known methods or systems. For example, the plant material or phytochemical composition (115) may be placed on a base or plate, within a bowl or cradle, or other holder (120), or simply suspended within the vacuum chamber (105) as would be convenient with stemmed plants and/or stemmed flowering plants (Not Shown). In some embodiments, holder (120) may define multiple apertures along at least a section of holder (120) for allowing phytochemicals emitted from material or composition (115) to travel to another area of the vacuum chamber (105).

It is contemplated that phytochemical collection may comprise simply allowing the vacuum chamber (105) vacuum/pressure to eventually via the non-actuated evacuation pump (110) equalize and return the vacuum chamber to ambient atmospheric pressure (210), and then collecting the extracted phytochemical from the interior of the vacuum chamber (105).

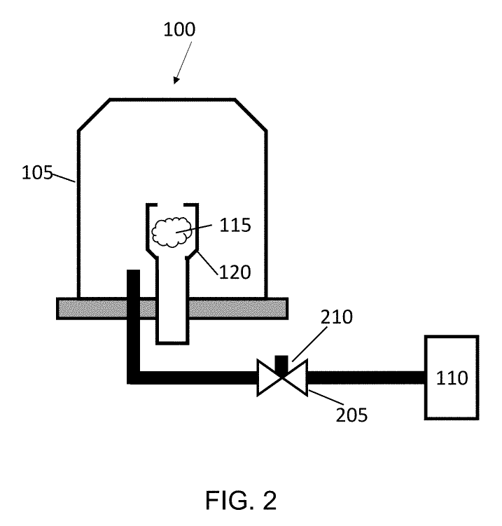

As depicted in FIG. 2, it is contemplated that a valve (205) may be included within the system to facilitate returning the chamber (105) to ambient atmospheric pressure (210).

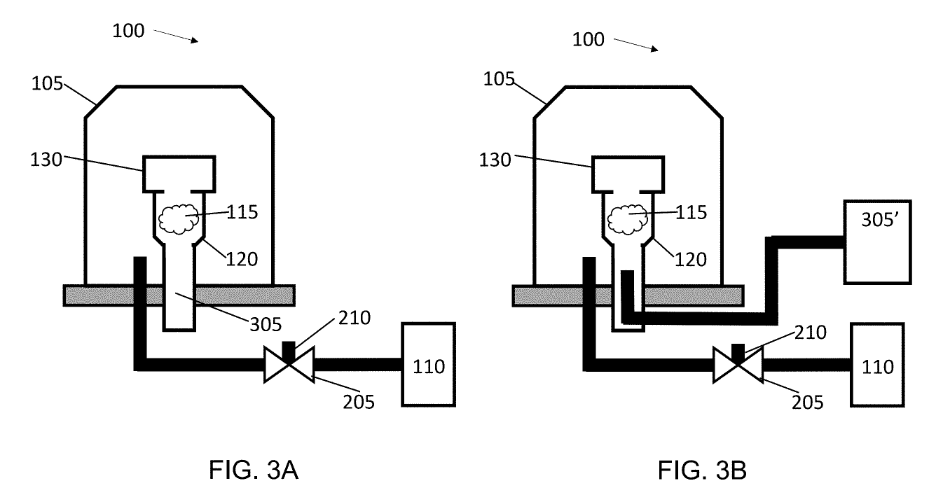

As depicted in FIG. 3A and FIG. 3B, it is contemplated that the method and system include a heat source (130) to increase the internal temperature of the vacuum chamber (105) and/or the temperature of the plant material or phytochemical composition (115) above ambient room temperature and thus increase volatilization of phytochemicals at a desired, selected, and/or provided partial vacuum.

In some embodiments, the temperature to which the heat source (130) increases the internal temperature of the vacuum chamber (105) and/or the temperature of the plant material or phytochemical composition (115) to enable volatilization of a phytochemical at a lower vacuum without causing pyrolysis of the plant material or phytochemical composition (115) is below 100° C. In some embodiments, the temperature is about 90° C., or about 80° C., or about 70° C., or about 60° C. or about 50° C., or about 40° C., or about 300 or about 20° C. or about 15° C. In other embodiments, the temperature to which the heat source (130) increases the internal temperature of the vacuum chamber (105) and/or the temperature of the plant material or phytochemical composition (115) to enable volatilization of a phytochemical at a lower vacuum without causing pyrolysis of the plant material or phytochemical composition (115) ranges from about 15° C. to about 230° C. For example, the temperature can be about 110° C., or about 120° C., or about 130° C., or about 140° C., or about 150° C., or about 160° C., or about 170° C., or about 180° C., or about 190° C., or about 200° C., or about 210° C., or about 220° C., or about 230° C.

It is contemplated that utilizing either controlled or explosive recompression of the chamber (105), the at least one phytochemical is collected via a collection chamber (305, 305′). In certain embodiments, the collection chamber (305, 305′) may be located within the vacuum chamber (305), or may be separate from and in fluid communication with the vacuum chamber (305′). It is contemplated that the system (100) include a valve (205) capable of controlled and/or explosive venting of the vacuum chamber (105) to the ambient atmosphere (210).

It is contemplated that utilizing explosive recompression of the vacuum chamber (105) may be one way that the at least one phytochemical is collected via the collection chamber (305, 305′).

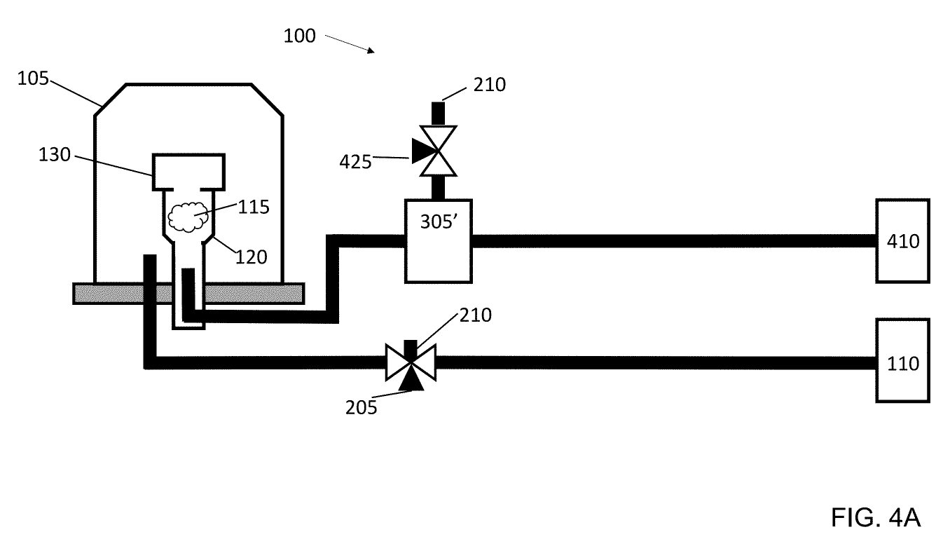

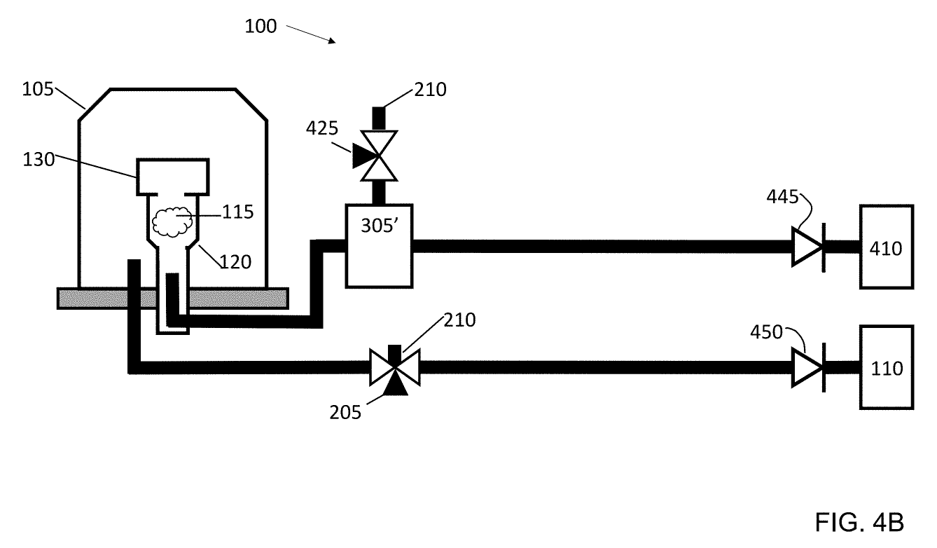

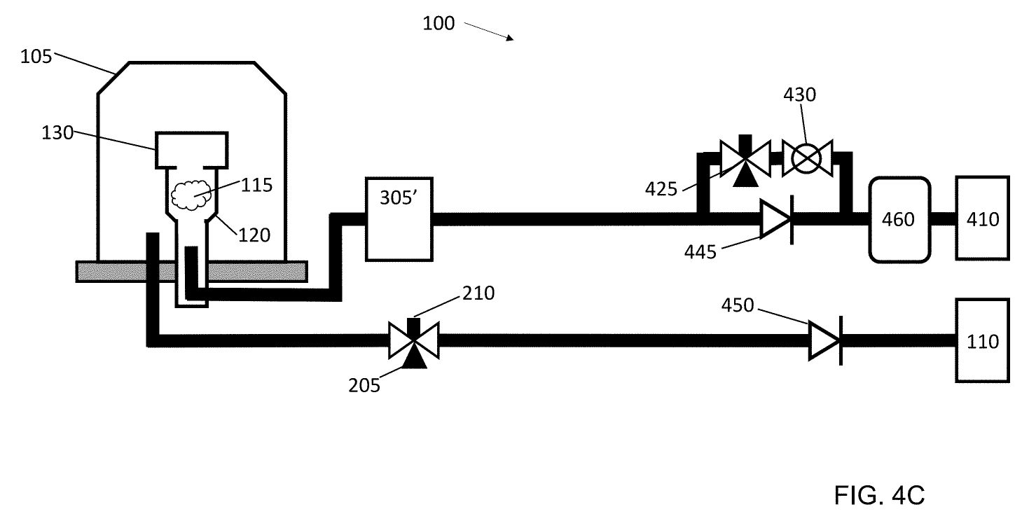

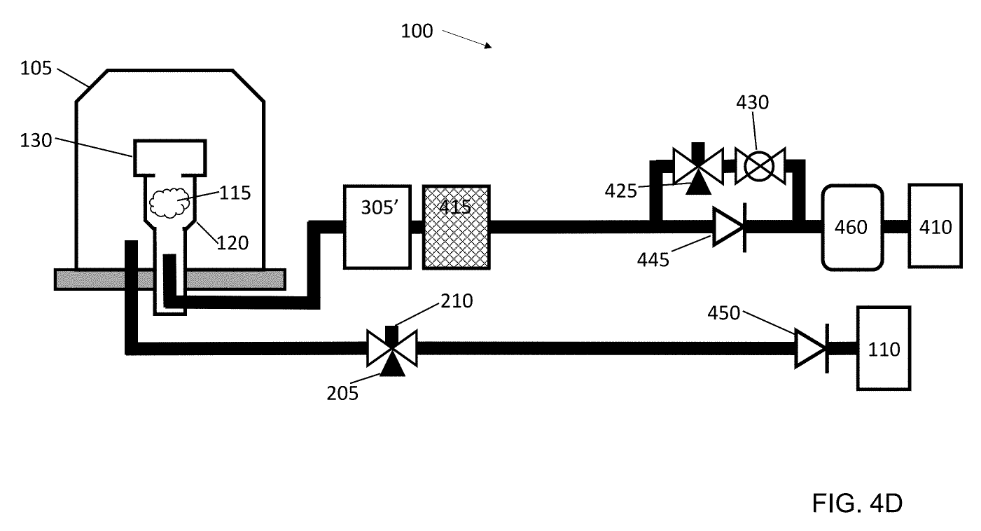

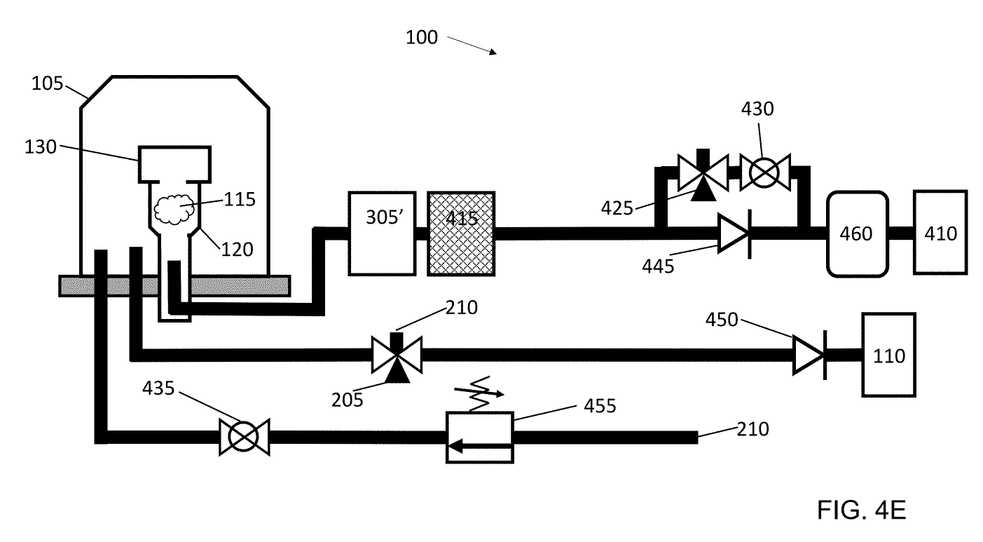

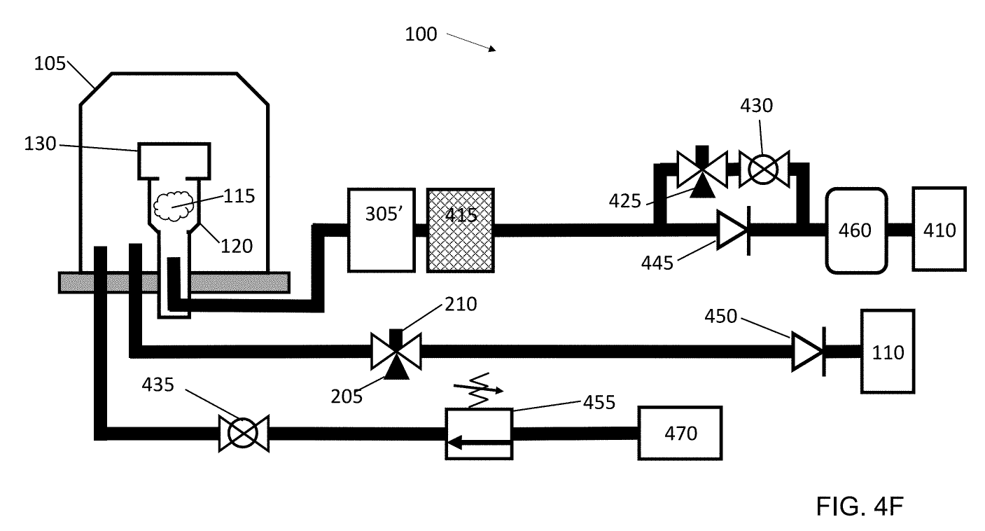

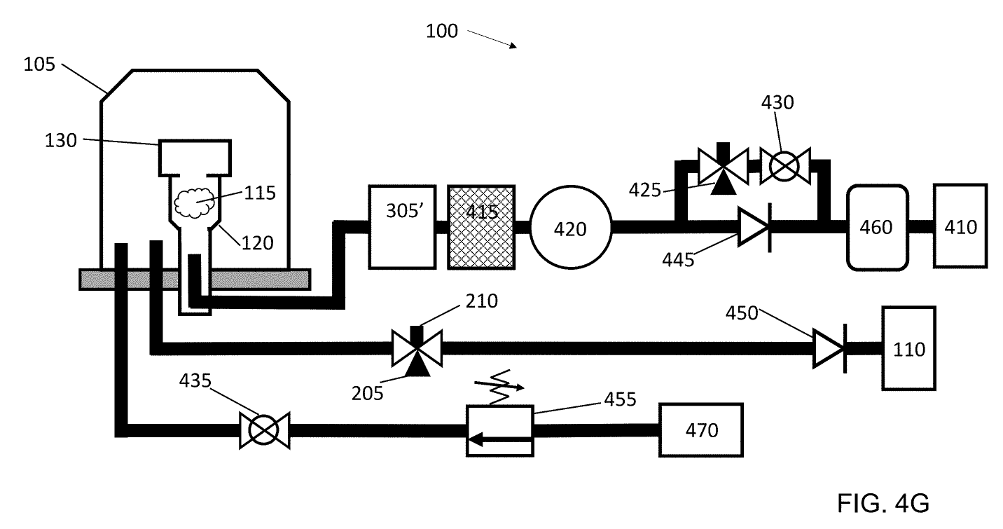

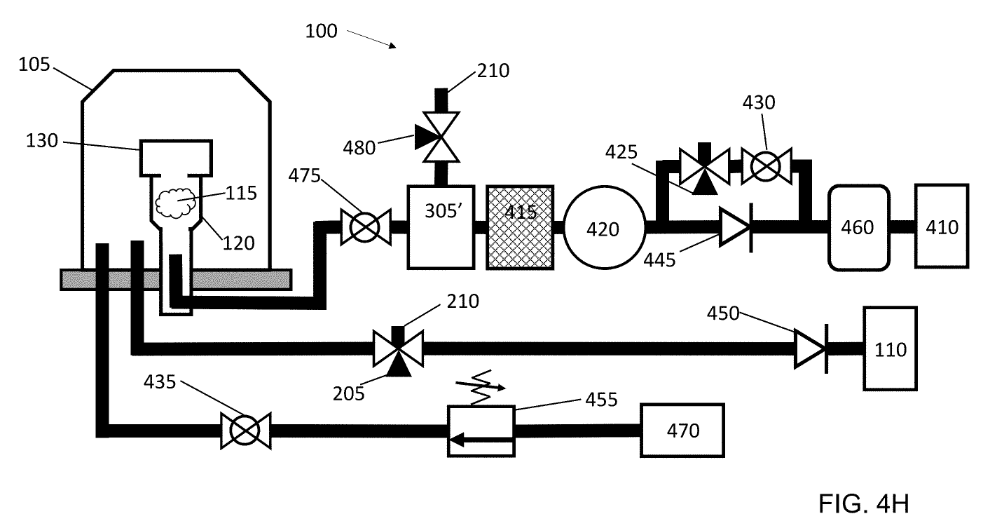

As depicted in FIG. 4A, it is contemplated that the inventive method and system (100) includes a second evacuation pump (410) capable of high-vacuum and high-velocity operation and in fluid communication with the collection chamber (305, 305′). It is contemplated that the system (100) include a valve (425) capable of controlled and/or explosive venting of the collection chamber (305, 305′) to the exterior ambient atmosphere (210).

As depicted in FIG. 4B, it is contemplated that the inventive method and system (100) includes at least one check valve (445) capable of high-vacuum and high-velocity operation in fluid communication with the collection chamber (305, 305′), and the second evacuation pump (410) to facilitate maintaining a selected and desired vacuum within the vacuum chamber (105) and/or collection chamber (305, 305′). It is contemplated that the inventive method and system (100) includes a second check valve (450) capable of high-vacuum and high-velocity operation in fluid communication with the vacuum chamber (105) and the evacuation pump (110) to facilitate maintaining a selected and desired vacuum within the vacuum chamber (105) and/or the collection chamber (305, 305′).

As depicted in FIG. 4C, it is contemplated that the inventive method and system (100) includes at least one ball or globe valve (430) capable of explosive operation or actuation in fluid communication with the collection chamber (305, 305′), a high-vacuum tank or reservoir (460), and the second evacuation pump (410). It is contemplated that an instantaneous or near-instantaneous vacuum source may be provided; the evacuation pump (110) providing and maintaining a selected or desired vacuum within the vacuum chamber (105) and/or collection chamber (305, 305′) for volatizing and/or precipitating a phytochemical from plant material or a phytochemical composition (115); and that via the at least one ball or globe valve (430), the high-vacuum tank or reservoir (460) holding a vacuum greater than the vacuum in the vacuum chamber (105) and/or the collection chamber (305, 305′) provided by the second evacuation pump (410). Once a selected period has elapsed after the selected or desired vacuum is achieved within the vacuum chamber (105) via the evacuation pump (110); the ball or globe valve (430) may be explosively opened to purge the system (100) and further collect at least one phytochemical (not shown) via the collection chamber (305 or 305′).

As depicted in FIG. 4D, it is contemplated that the inventive method and system (100) includes at least one trap or filter including a plurality of variably configured apertures (415) in fluid communication with the collection chamber (305, 305′), the high-vacuum tank or reservoir (460), and the second evacuation pump (410). As previously described above regarding FIG. 4C, utilizing the evacuation pump (110) to provide and maintain a selected or desired vacuum within the vacuum chamber (105) and/or collection chamber (305, 305′) for volatizing and/or precipitating a phytochemical from plant material or a phytochemical composition (115); and that via the at least one ball or globe valve (430), the high-vacuum tank or reservoir (460) holding a vacuum greater than the vacuum in the vacuum chamber (105) and/or the collection chamber (305, 305′) provided by the second evacuation pump (410); an instantaneous or near-instantaneous vacuum source is provided. Once a selected period has elapsed after the selected or desired vacuum is achieved within the vacuum chamber (105) via the evacuation pump (110); the ball or globe valve (430) may be explosively opened to purge the system (100) and further collect at least one phytochemical (not shown) via the collection chamber (305 or 305′) and/or via the trap or filter (415).

It is contemplated that the trap or filter (415) may be remote from, and/or internal to or integral with (not shown), the collection chamber (305, 305′). It is also contemplated that the collection chamber (305, 305′) and/or the trap or filter may be cooled to a temperature below the temperature of the vacuum chamber (105) to more effectively and efficiently collect a desired or selected phytochemical.

As depicted in FIG. 4E, it is contemplated that the inventive method and system (100) includes at least one variable vacuum/pressure regulator (455) in fluid communication with: the vacuum chamber (105), a second ball or globe valve (435), in fluid communication with the external ambient atmosphere (210). Similar to as previously described above regarding FIG. 4C and FIG. 4D; utilizing the evacuation pump (110) to provide and maintain a selected or desired vacuum within the vacuum chamber (105) and/or collection chamber (305, 305′) for volatizing and/or precipitating a phytochemical from plant material or a phytochemical composition (115); and that via the at least one ball or globe valve (430), the high-vacuum tank or reservoir (460) holding a vacuum greater than the vacuum in the vacuum chamber (105) and/or the collection chamber (305, 305′) provided by the second evacuation pump (410); an instantaneous or near-instantaneous vacuum source is provided. Once a selected period has elapsed after the selected or desired vacuum is achieved within the vacuum chamber (105) via the evacuation pump (110); the ball or globe valve (430) may be explosively opened to purge the system (100) and further collect at least one phytochemical (not shown) via the collection chamber (305 or 305′) and/or via the trap or filter (415). It is contemplated that to prevent any extracted phytochemical from undesired reverse travel within the system (100), the second ball or globe valve (435) and the variable vacuum/pressure regulator (455) may be activated in a controlled manner to de- or re-compress the vacuum chamber (105) and/or collection chamber (305, 305′).

As depicted in FIG. 4F, in one embodiment of the inventive method and system, a pressurized gas or air reservoir (470) is provided and in fluid communication with the variable vacuum/pressure regulator (455), wherein instead of recompressing the vacuum chamber (105) to external ambient atmospheric pressure (210), actuation of the second ball or globe valve (435) explosively compresses and/or pressurizes the vacuum chamber (105) to the approximate gas or air pressure within the pressurized reservoir (470) dependent upon the setting of the variable vacuum/pressure regulator (455), thus more efficiently and effectively removing any extracted phytochemical from the surface of the material or composition, and/or from the interior of the system (100). Explosive compression of the chamber (110) to a pressure above ambient air pressure (210) further facilitates collection of the at least one phytochemical by stripping or dislodging and collecting the volatized and/or precipitated at least one phytochemical.

As depicted in FIG. 4G, in one embodiment of the inventive method and system (100) includes a known distillation and/or vacuum distillation step (420) of the phytochemical collected to substantially remove any solvent, ballast, fat, wax, carbohydrate, protein, sugar, and/or terpene therefrom the plant material or phytochemical composition (115).

As depicted in FIG. 4H, in one embodiment of the inventive method and system (100) includes valves (475, 480) in fluid communication with the collection chamber (305, 305′) and the ambient atmosphere (210). It is contemplated that with valve (475) open, valve (480) closed, and the vacuum chamber (105) under at least a partial vacuum; at least one phytochemical will collect in the collection chamber (305, 305′). As desired or selected, valve (475) is closed and valve (480) opened to more efficiently and effectively collect the at least one phytochemical via the filter or trap (415).

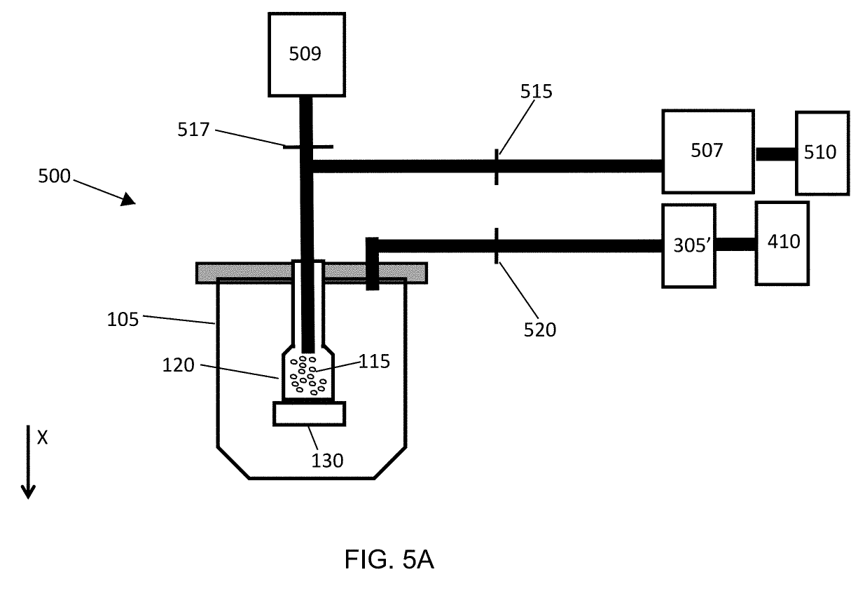

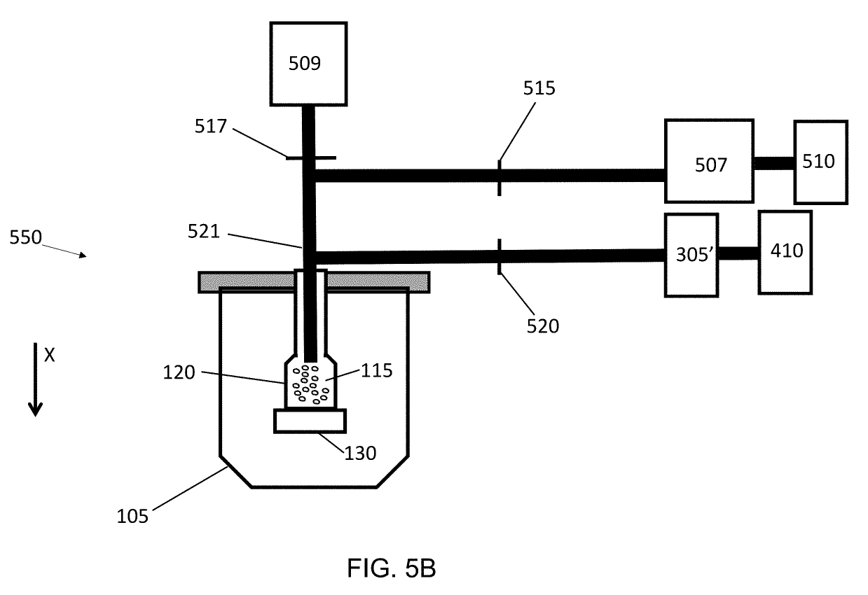

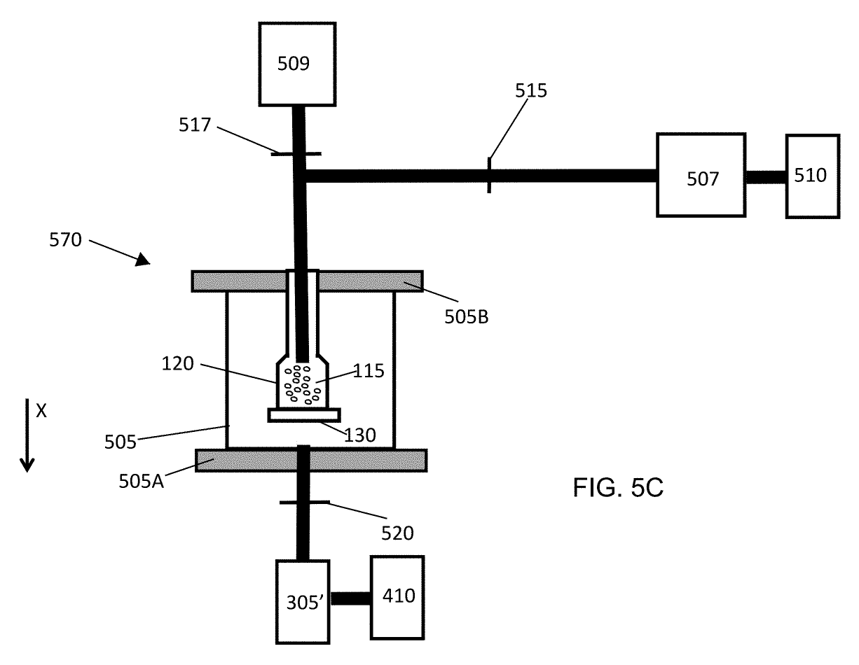

FIGS. 5A, 5B, and 5C show embodiments with evacuation pumps (410) and (510), however, said evacuation pumps (410) and (510) can be combined into one evacuation pump and/or further evacuation pumps may be in fluid communication with evacuation pumps (410) and (510).

As depicted in FIG. 5A, “X” denotes the general direction of gravity in one embodiment of the inventive method and system (500). Although specified in this embodiment, all other embodiment may also share this direction of gravity or may be placed in different orientations and arrangements vis-à-vis the direction of gravity, particularly vacuum chamber (105), holder (120), and heat source (130).

Dispenser (509) is arranged to provide the plant material or phytochemical composition (115) for vaporization. Holder (120) accepts said material or composition (115). Dispenser (509) and holder (120) may be isolated from each other via valve (517) (e.g., a gate valve) or an “air lock”, as described in more detail in (combinable) aspects of the below-described embodiments. Said material or composition (115) may be gravity assisted in traveling from dispenser (509) to holder (115) and/or rely on a pressure differential between dispenser (509) or air lock (not shown) and vacuum chamber (105).

ABV (already been vaped) plant material or phytochemical composition (not shown) may be collected (e.g., “suctioned up”) by ABV collector (507). ABV collector (507) may utilize a pressure differential, such as in this embodiment, and/or gravity, as shown in other embodiments.

Valves (515) and (520) respectively isolate ABV collector (507) and collection chamber (305′) from the vacuum chamber.

As depicted in FIG. 5B, it is contemplated that the “plumbing” (e.g., pipes and valves) in one embodiment of the inventive method and system (550) is slightly different than method and system (500) with dispenser (509), ABV collector (507), and collection chamber (305′) all being (potentially) in fluid communication via section (521) that is outside of vacuum chamber (105), even if one or more of the valves (517, 515, 520) would typically be closed for isolation.

As depicted in FIG. 5C, it is contemplated in one embodiment of the inventive method and system (570) to utilize at least gravity for guiding the vapor (that contains one or more phytochemicals) that is emitted from plant material or composition (115) towards collection chamber (305′). Vacuum chamber (505) may be (partially) defined by two substrates (505A, 505B) on either end.

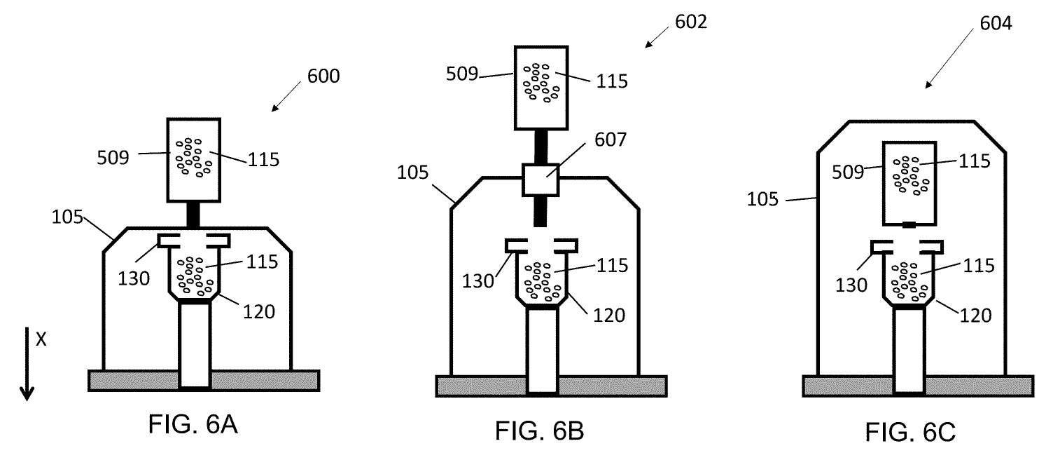

FIGS. 6A, 6B, and 6C omit showing evacuation pumps for the sake of focusing on other embodiment aspects. Vacuum chamber (105), as shown in FIGS. 6A, 6B, and 6C, may be in fluid communication with one or more pumps, as shown in the other embodiments.

As depicted in FIGS. 6A, 6B, and 6C, it is contemplated that the inventive method and system (600, 602, 604) arrange dispenser (509) inside or outside vacuum chamber (105). It is contemplated that air lock (607) may include two valves (not shown). A “top” valve for admitting the material or composition (115) into air lock (607) at or near, for example, atmospheric pressure. After closing the top valve, air lock (607) may then be brought to a low, medium, or high vacuum via an evacuation pump (not shown). Then, the “bottom” valve of air lock (607) is opened to allow the material or composition (15) to be dispensed onto/into holder (120) which is under the same or similar quality of vacuum. This ensures a “smooth” journey of the material or composition (115) from dispenser (509) to holder (120), even if dispenser (509) and holder (120) are, at times, under vastly different pressures (e.g., atmospheric pressure vs. medium vacuum) when isolated from each other.

Air lock (607) may be arranged to be fully within, fully outside, or partially within vacuum chamber (105).

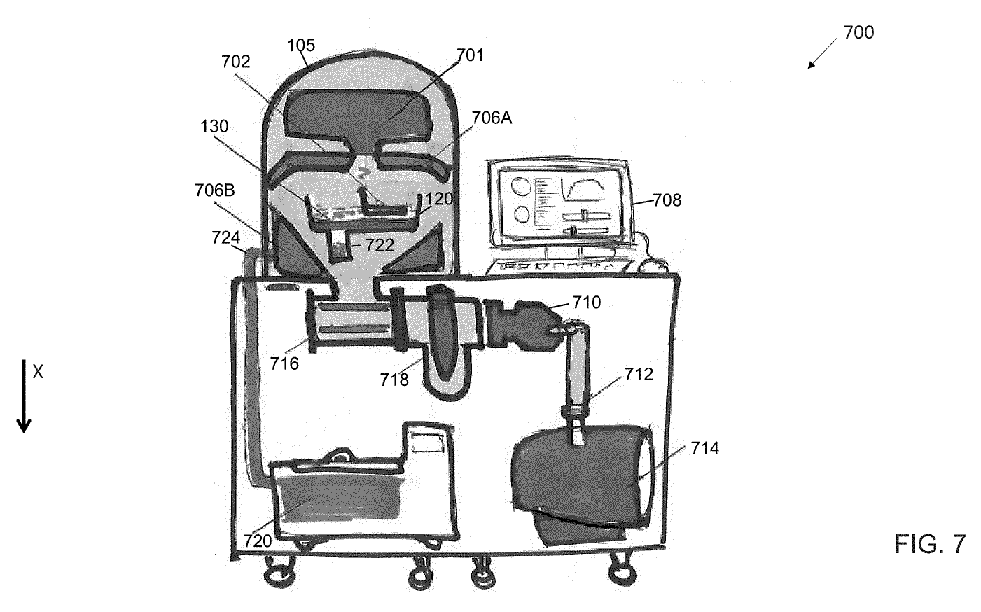

As depicted in FIG. 7, it is contemplated that the inventive method and system (700) includes a vacuum chamber (105) that houses dispenser (701), stirrer (702), collectors (706A, 706B), holder (120), and heat source (130). Other embodiments may include more or fewer elements within chamber (105) than what is shown in FIG. 7.

The inventive system and method (700) further includes controller (708), high-vacuum evacuation pump (710) (e.g., a turbomolecular pump), vacuum plumbing (712) (e.g., pipes and valves) arranged between pumps (710) and “rough” evacuation pump (714), which creates a vacuum on the outlet side of the high-vacuum evacuation pump (710).

The inventive system and method (700) further includes cold trap collector (716), protective cold trap (718), chiller (720), ABV collector (722), and coolant lines (724) from chiller (720).

Controller (708) may be a programmable logic controller. Controller (708) controls various subsystem (dispensing, stirring, heating, cooling, vacuuming, collecting, and the like) and actuators thereof for system and method (700) as well as possibly providing a user interface for adjusting and setting variables such as vacuum pressure values and times, heating temperature and times, cooling temperatures and times, and the like.

Cold trap collector (716) collects at least one phytochemical extract along with collectors (706A) and (706B). Protective cold trap (718) collects other items that flow downstream of collector (716) and may be used to also collect a phytochemical extract.

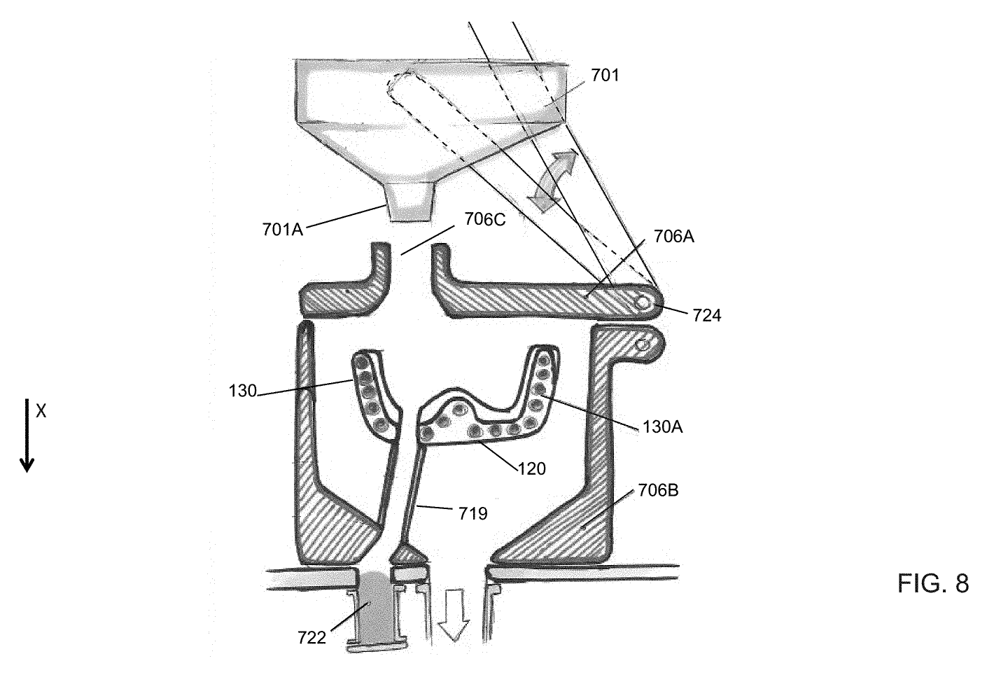

As depicted in FIG. 8, it is contemplated that dispenser (701) may mechanically couple to a section of collector (706A) via shoot (701A) and aperture (706C). Collector (706A) may be rotatable via pivot (724) for allowing, among other things, access to heat source (130) and collectors (706A, 706B).

ABV material or composition (not shown) may be guided to ABV collector (722) via a paddle (not shown) guiding the ABV material or composition towards shoot (719), which is in fluid communication with both holder (120) and collector (722). As previously stated, ABV collector may also (or instead) rely on a pressure differential to suck up the ABV material or composition from holder (120) to ABV collector (722). Shoot (719) may be a glass tube.

As shown in FIG. 8, aperture (130A) of heat source (130) allows for the ABV material or composition to feed shoot (719) when said material is paddled or suctioned up.

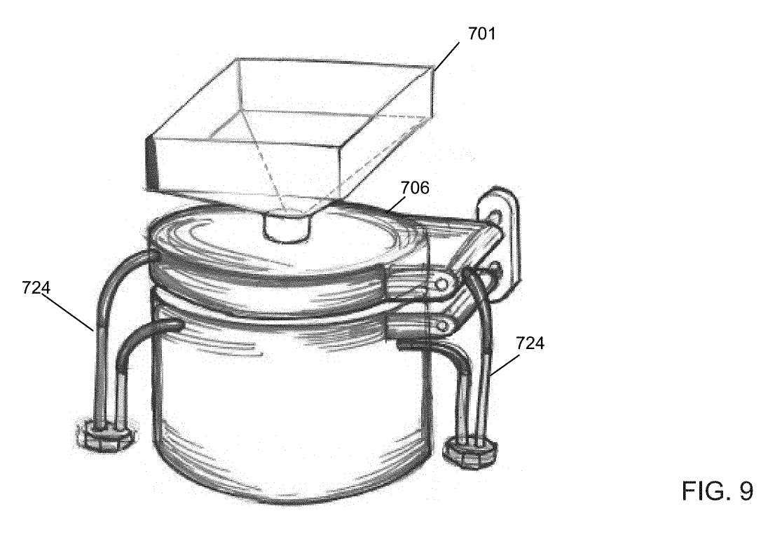

As depicted in FIG. 9, it is contemplated that collector (706) may be cooled via coolant that is circulated via coolant lines (724), which are in fluid communication with collector (706) and chiller (720) (shown in FIG. 7).

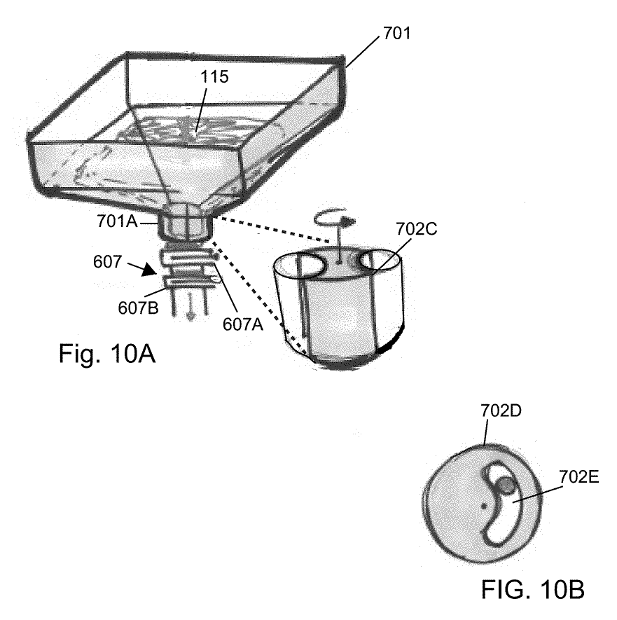

As depicted in FIG. 10A, it is contemplated that dispenser (701) includes rotating mechanism (702C) arranged within shoot (701A) for feeding material or composition (115) into air lock (607), which includes valves (607A) and (607B). Said feeding may be gravity assisted, rely on pressure differentials between air lock (607) and dispenser (701), or utilize both gravity and pressure differentials. As depicted in FIG. 10B, bottom face (702D) of shoot (701A) defines aperture (702E) which is alternately blocked as rotating mechanism (702C) rotates, thus allowing material or composition 115 to fall (and/or be sucked) through.

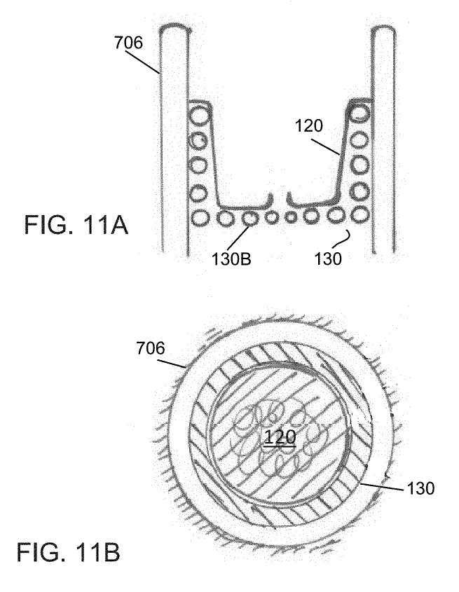

FIGS. 11A (side view) and 11B (top view) depict an embodiment example of holder (120), heat source (130), and collector (706). Holder (120) may include a stirrer, which is not shown in these figures. Heat source (130) includes a plurality of heating elements (130B), which may emit heat as a whole and/or (controlled) sub-groups of one or more heating elements (130B). In this embodiment, it is contemplated that collector (706) abuts a periphery section of heat source (130). Heating elements (130B) may be, for example, coils (e.g., resistive heating elements) or bulbs (e.g., bulbs that emit IR energy).

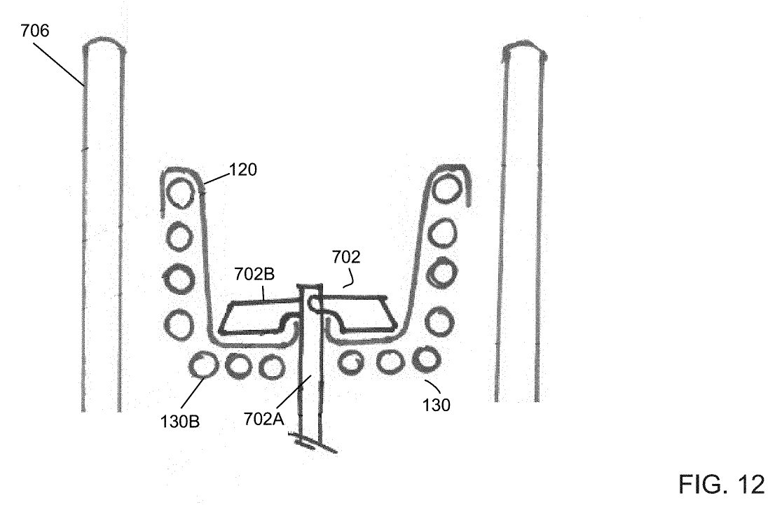

As depicted in FIG. 12, it is contemplated that a gap may exist between the periphery of heat source (130) and collector (706). It is further contemplated, that stirrer (702) may include shaft (702A) operably coupled to blades (702B). A motor or other turning mechanism (not shown) rotates shaft (702A), which rotates blades (702B) for stirring a plant material or phytochemical composition before, during, and/or after heating. Stirring helps evenly vaporize said material or composition and may help dispose said material or composition down a shoot (not shown) for disposing an ABV material or composition (not shown).

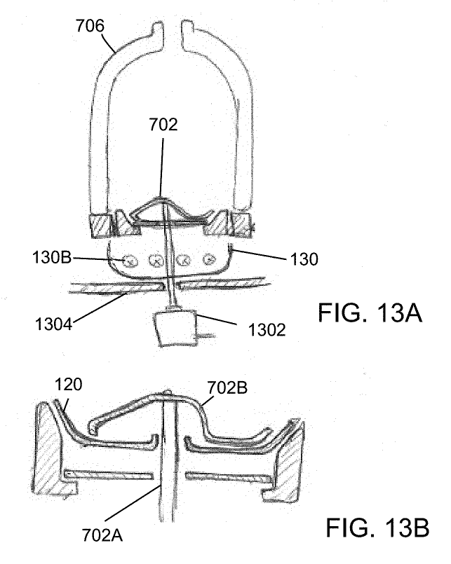

As depicted in FIGS. 13A and 13B, it is contemplated that motor (1302) may reside on one side of substrate (1304) and collector (706), blades (702B), holder (120) and heat source (130) on another. Substrate (1304) may delineate a vacuum chamber (not show) such that motor (1302) resides outside the vacuum chamber.

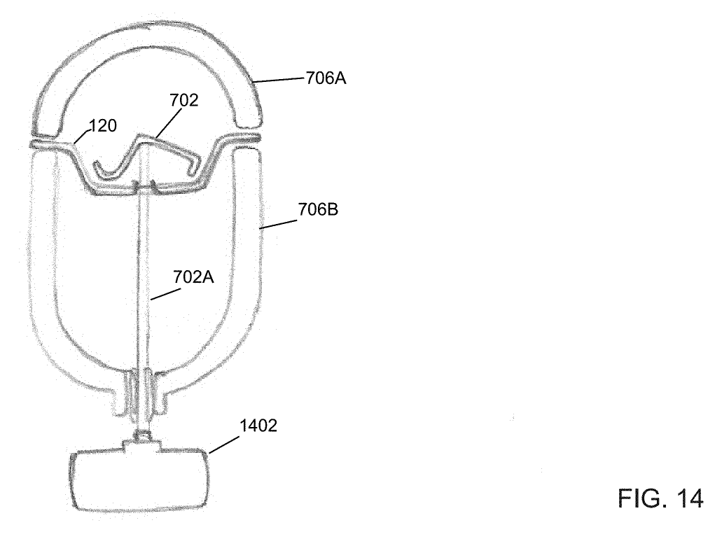

As depicted in FIG. 14, it is contemplated that small collector (706A) is arranged on one side of holder (120) and large collector resides on another side of holder (120). Motor (1402) turns stirrer (702), as described above.

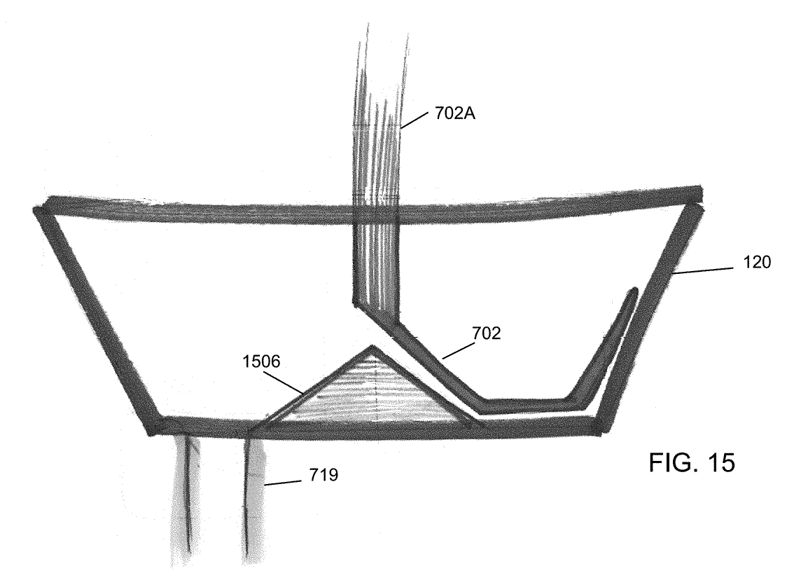

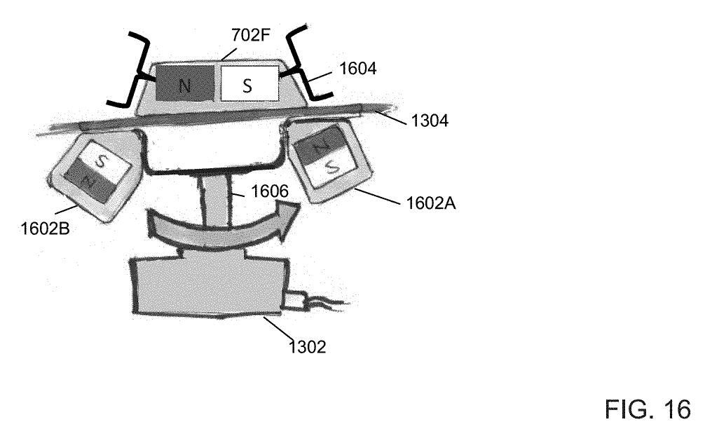

As depicted in FIG. 15, it is contemplated that holder (120) may define raised feature (1506) for dispersing the material or composition within holder (120). Stirrer (702) may rotate around a rotational axis that is coaxial with shaft (702A).

As depicted in FIG. 16, it is contemplated that the stirring mechanism may reside on different sides of substrate (1304), which may define a periphery of a vacuum chamber (not shown) such that motor (1302) resides outside the vacuum chamber, but embodiments may also include motor (1302) inside a vacuum chamber. Motor (1302) is coupled to one or more motor magnets (1602A) and (1602B) via shaft (1606). Stirrer (702) includes housing (1604), which may encapsulate stirrer magnet (702F) and may be coupled to blades (702B). Housing (1604) may have a low-friction coating such as Teflon.

Due to magnetic attraction, motor (1302) will rotate stirrer (702) when motor (1302) rotates motor magnets (1602A, 1602B). This embodiment avoids potentially placing a shaft into the vacuum environment in the case that substrate (1304) defines a periphery of a vacuum chamber. Substrate 1304 may be a quartz material, among other possibilities.

Such other possibilities for the material of substrate 1304 include but are not limited to glass, ceramic, or any other inert materials, capable of providing a benefit of highly efficient heat transfer to the material to be extracted. One of many benefits of efficient heat transfer is prevention of off-gassing in the extraction process. Other benefits of the combination of highly efficient heat transfer combined with continuous low pressure include the ability for terpenes and cannabinoids to volatilize and enter the extraction streams at the comparatively very low temperatures, resulting in minimal or no degradation of the terpenes and cannabinoids. This highly efficient, low-temperature, continuous low-pressure approach also results in maximal to complete quantitative recovery of terpenes and cannabinoids, to a degree that has not been observed and is simply not possible with solvent-based extraction systems. When coupled with a continuous feed capability under continuous vacuum, as discussed herein, the system also becomes fully scalable.

Efficient, rapid, and uniform transfer of heat is also achieved via the design and function of the system wherein the material to be extracted is dropped through the top valve onto a heated plate which is already at the desired temperature, and stirred in a dynamic heating system which flash-heats the material, thus combining speed of heating with extremely uniform distribution of the heat throughout the material, rather than a gradual and unevenly distributed heating of the material. As would be appreciated by a person of skill in the art, terpenes and cannabinoids having very precise temperature/pressure vaporization points will volatilize only as uniformly from plant material as the material itself is uniformly exposed to conditions of temperature and (low) pressure. Accordingly, the inventive systems and methods described herein achieve the extremely high fidelity in extraction efficiency and profiles, comparing the composition of the final extract to the composition of the original plant material, in large measure due to the great uniformity and speed with which the plant material is all exposed to the right conditions of temperature and pressure to cause the volatilization to occur.

In FIGS. 5A-C, 7, 8 and 17 to 20A-F, among other possible embodiments, it is contemplated to continuously extract at least one phytochemical by feeding a holder and/or vacuum chamber the plant material or phytochemical composition and collecting at least one of the processed material or composition and the at least one extracted phytochemical. This process may be repeated (e.g., feeding and collecting) while maintaining or substantially maintaining an established vacuum, particularly when the holder and vacuum chamber are essentially one of the same, although continuous extraction embodiments are not limited to such a feature, as shown, for example, in FIGS. 5A-C, 7, and 8.

A “holder” may partially or substantially define a vacuum chamber and vise-versa. Thus, embodiments may identify a section as both as both the vacuum chamber and holder, which will be referred to as “holder (105, 120)” or “vacuum chamber (105, 120)”. Further still, a “rotatable holder” may combine the functionalities of a holder and stirrer (and perhaps the vacuum chamber) by rotating and thereby moving a material or composition's position within the rotatable holder. Each of these features, alone or in various combinations, allow for a degree of automization that was heretofore unknown in the vacuum extraction arts. Continuous extraction embodiments may also break a vacuum without suffering much of a time penalty for re-establishing the vacuum when holder (105, 120) has a relatively small volume (e.g., 1 to 3 liters).

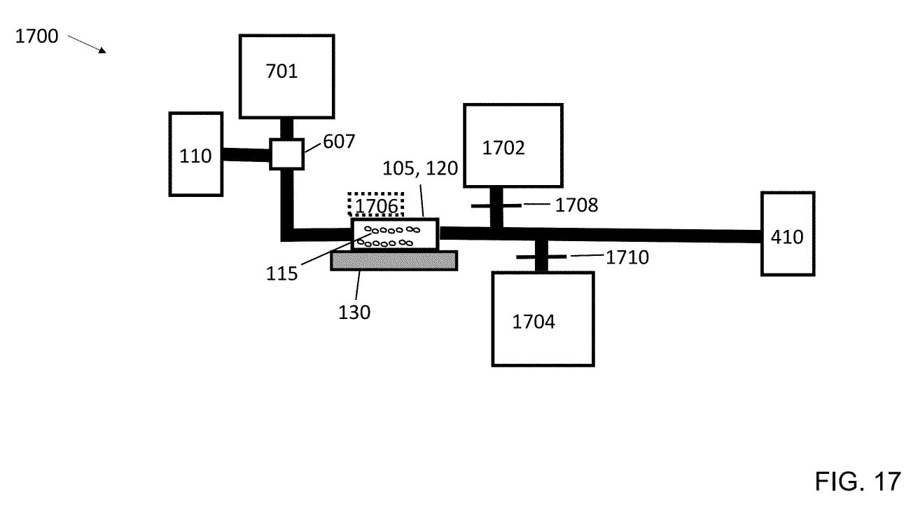

As depicted in FIG. 17, it is contemplated that inventive method and system (1700) includes dispenser (701) feeding material or composition (115) to airlock (607), as described for FIG. 6B. In other words, dispenser (701) may feed airlock (607) material or composition (115) at atmospheric pressure or other “first pressure”. The top valve (not shown) of airlock (607) is then closed and evacuation pump (110) brings airlock (607) down to a vacuum near or at the same vacuum quality of holder (105), which may be established by evacuation pump (410). The bottom valve (not shown) then opens for allowing material or composition (115) to enter holder (105, 120), which is operably coupled to heat source (130).

Collector (1702) collects oil and/or vapor containing at least one phytochemical composition and ABV collector (1704) collects an ABV (or “already been processed” or “ABP” for non-vaporizer embodiments) material or composition. Collector (1702) and/or ABV collector (1704) may be isolated from an established vacuums by airlocks and/or valves (1708) and (1710).

Vibration element (1706) may be operably coupled to holder (105, 120) to induce or supplement moving material or composition (115) from the dispenser side of holder (105, 120) to the ABV collector side of the holder (105, 120). Holder (105, 120) may vibrate or “shake” laterally, vertically, or a combination thereof. Vibration elements may be, among other things, vibration actuators, vibration motors, Eccentric Rotating Mass (ERM) motors, or Linear Resonance Actuators (LRA).

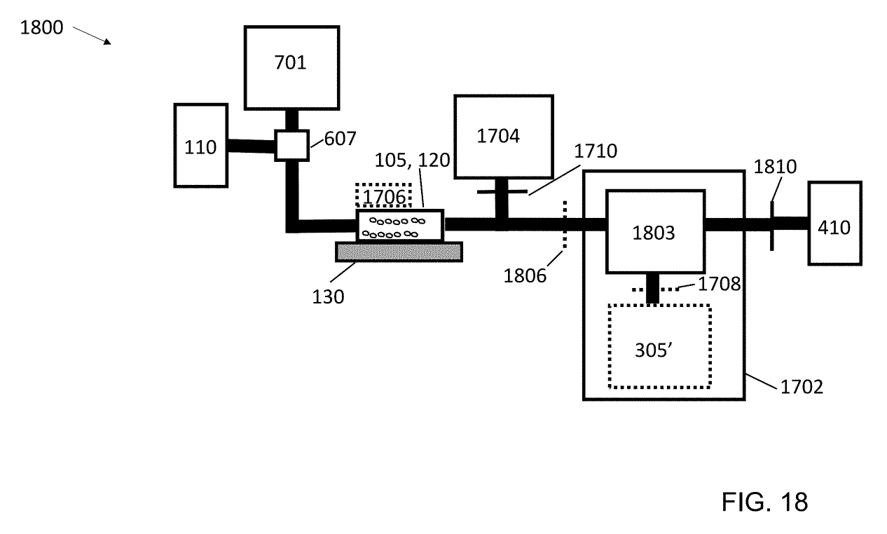

As depicted in FIG. 18, it is contemplated that the inventive method and system (1800) may include collector (1702) that includes at least cold trap (1803) for collecting the at least one phytochemical, typically in a condensed vapor or oil form. Collector (1702) may further include collection chamber (305′), in which the oil is collected from cold trap (1803). Valves (1806) and (1708) may respectively isolate collector (1702) and collection chamber (305′). For example, valves (1806) and (1810) may be closed to allow for the removal of cold trap (1803) for collection oil therefrom without breaking a vacuum of holder (105, 120).

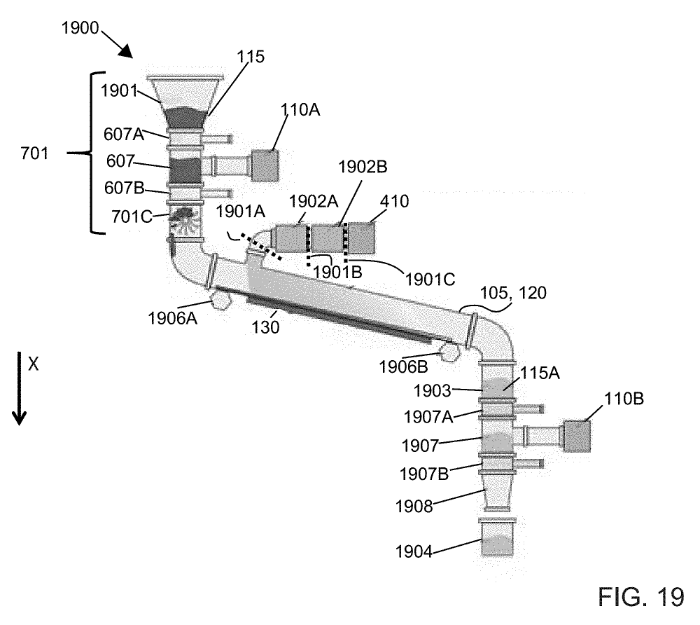

As depicted in FIG. 19, it is contemplated that the inventive method and system (1900) may rely on one or more of gravity, vacuum/pressure differential, temperature differential, vibration, and a rotating mechanism for achieving continuous extraction. Dispenser (701) includes hopper (1901), air lock (607) with valves (607A) and (607B), and rotating mechanism (701C) for dispensing material or composition (115) into vacuum chamber (105, 120). Air lock (607) is fluidly connected to vacuum pump (110A).

As shown, heater (130) is arranged between vibration elements (1906A) and (1906B). Vibration elements (1906A) and (1906B) may operate continuously, intermittently, and/or alternately. For example, element (1906A) may first operate and element (1906B) may then begin operation some time thereafter, either temporally overlapping or sequentially operating after element (1906A) has turned off. Alternative embodiments may include only one vibration element and three or more vibration elements.

Collection chambers (1902A) and (1902B) may be isolated by one or more valves (1901A), (1901B), and (1901C) from vacuum chamber (105, 120) and from pump (410). Collection chambers (1902A) and (1902B) collect the at least one composition and may be removed from system (1900). In some embodiments, such removal does not disrupt the operation of system and method (1900), including, for example, substantially maintaining an established vacuum within vacuum chamber (105, 120) when one or both collection chambers (1902A) and (1902B) are removed.

ABV material or composition (115A) is collected in holder (1903), which feeds airlock (1907), which includes valves (1907A) and (1907B), and is fluidly connected to vacuum pump (110B). Discharge section (1908) feeds holder (1904). In some embodiments, holder (1904) may be mechanically connectable to discharge section (1908).

In use, large quantities of feedstock (115) can be fed into hopper (1901) at once, and topped up at any time. The feedstock (115) passes through air lock (607) where air is pulled out of the feedstock. The feedstock (115) is dosed to feed a consistent layer of material into the vacuum chamber (105, 120). Vibration elements (1906A and 1906B) ensure the layer of material moves through the vacuum chamber (105, 120) at a consistent rate, and a heat source (130) vaporizes the material passing over it. The vapor is pulled out of the vacuum chamber (105, 120) by vacuum pump (410). Vapor is collected through vapor collection chambers (1902A and 1902B) with the option of selective fractioning. ABV material (115A) is held in holder (1903), passed out of air lock (1907), and expelled to holder (1904).

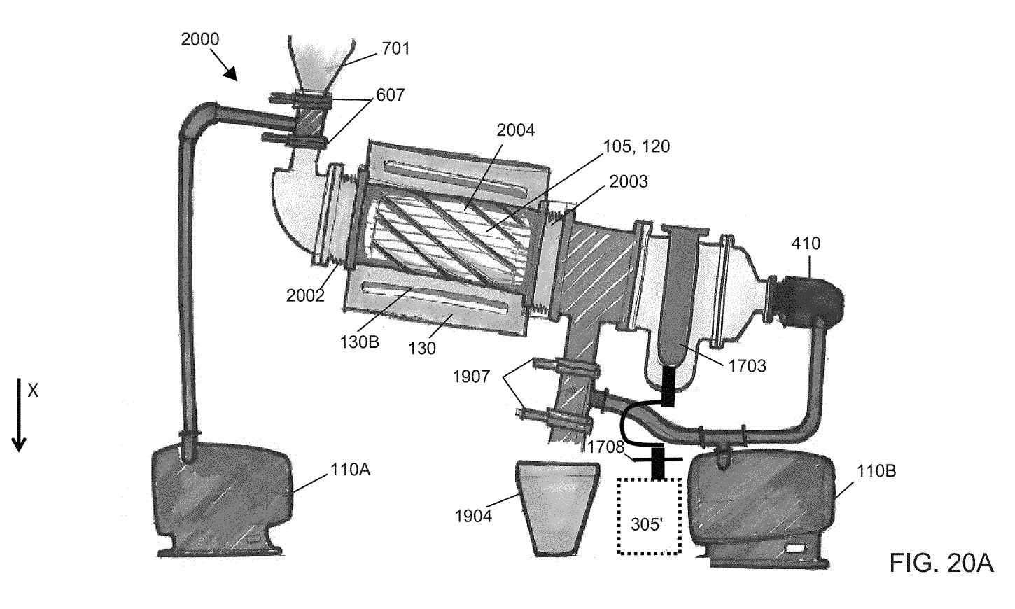

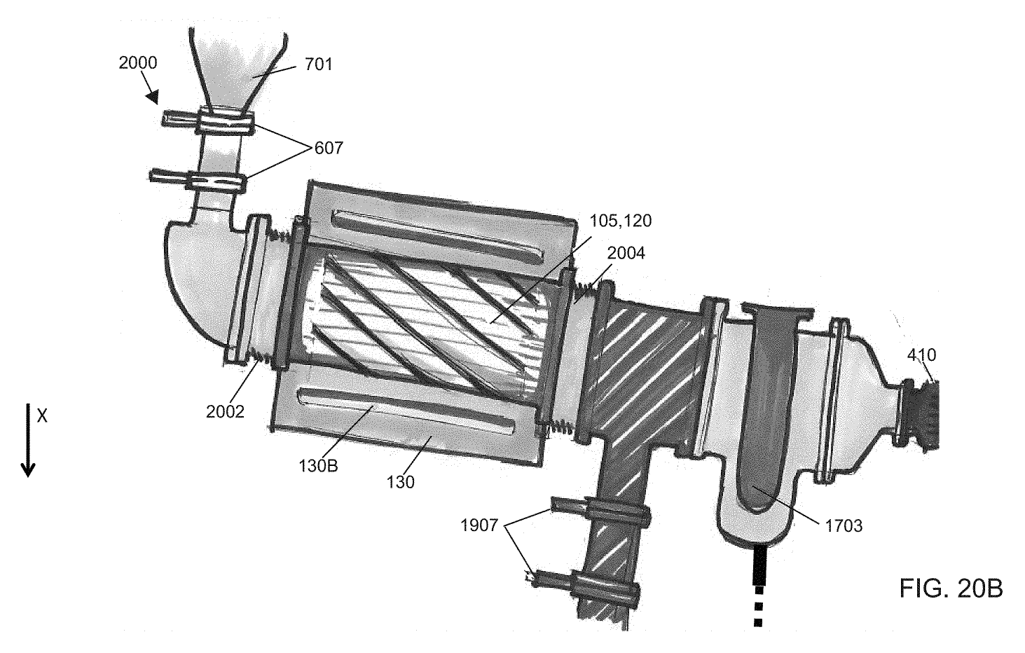

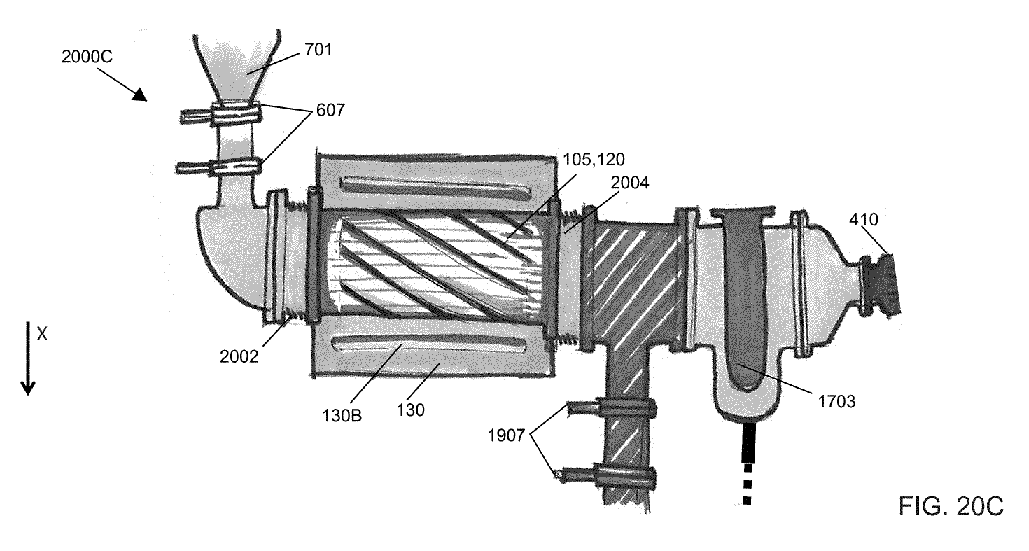

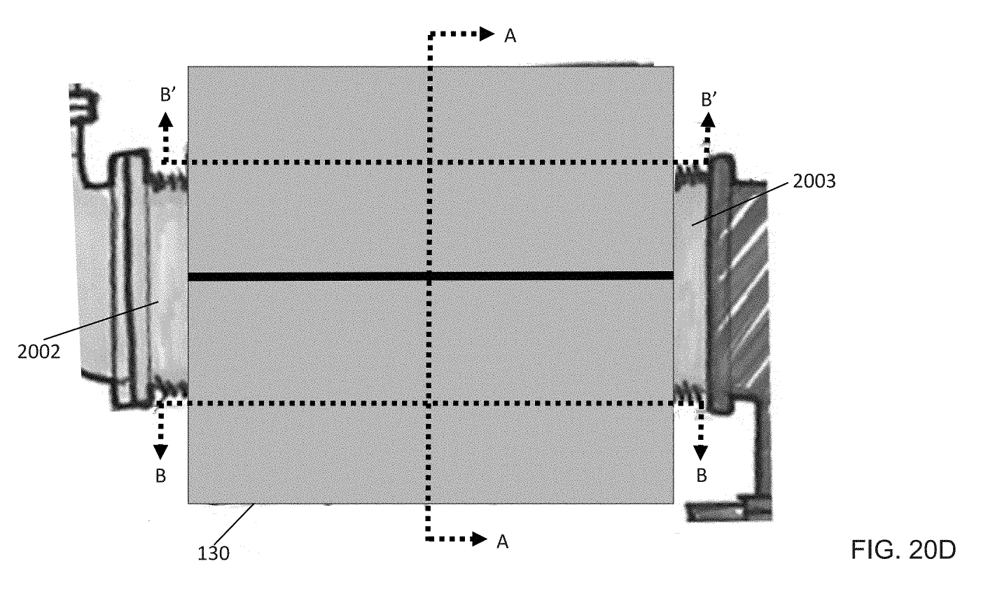

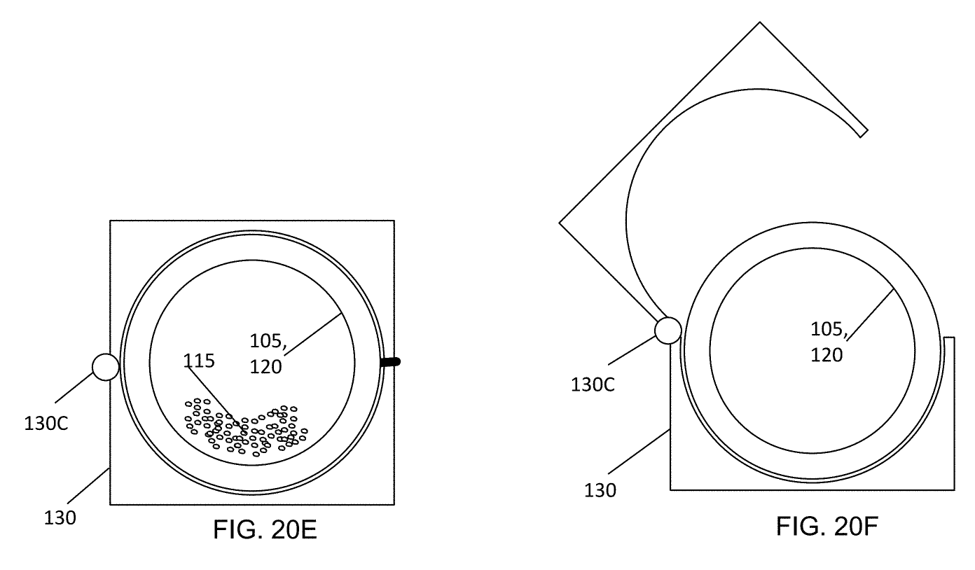

As depicted in FIGS. 20A to F, it is contemplated that the inventive method and system (2000) may essentially combine a stirrer (e.g., a rotating mechanism), a vacuum chamber, and a holder into holder (105, 120). One skilled in the art is aware of a number of techniques for driving the rotation of holder (105, 120), including elements arranged within and/or exterior to rotatable connections (2002) and (2003) and/or rotatable holder (105, 120). The figures also show dispenser (701, air lock (607) with gate valves and fluidly connected to pump (110A) via plumbing and valves, air lock (1907) with gate valves and fluidly connected to pumps (410A and 110B), holder (1904), cold trap (1703), and collection chamber (305′).

As shown, rotatable holder (105, 120) is cylindrical with internal, raised grooves (2004) on its inner surface. Alternative embodiments may include smooth internal surfaces, recessed grooves, and/or other raised and/or recessed features that are defined on the inner surface of a rotating holder. Holder (105, 120) is coupled, respectively on each side, to rotatable connections (2002) and (2003) and thereby defining one or more rotary joints. In FIG. 20C, holder (105, 120) is arranged substantially orthogonal to the direction of gravity rather than at a non-orthogonal orientation, as shown in FIGS. 20A and 20B.

Rotatable holder (105, 120) may include a transparent or semi-transparent material such as quartz, which is substantially “invisible” or transparent to wavelength(s) emitted by heat source (130). This may allow for an efficient transfer of the emitted energy to the material/composition.

Heat source (130) is shown as a cut-way, cross section in FIGS. 20A-C, 20E, and 20F and includes heating element (130B), which may be, for example, and IR bulb. FIGS. 20A-C show a cut-away, cross section of heat source (130) along lines B and B′ (i.e., the area between lines B and B′ of FIG. 20D is cut-away to show rotatable holder (105, 120). FIG. 20D is a side view of, among other things, heat source (130). FIGS. 20E and 20F are cross sectional views along line A of FIG. 20D of a respective holder (105, 120) and/or heat source (130) and pivot 130C (e.g., at least one of a heat source and holder is capable of lateral movement).

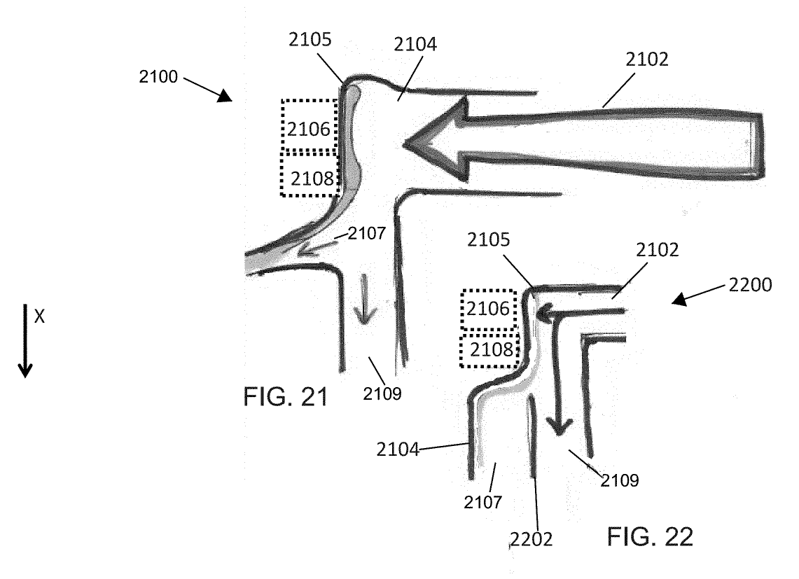

As depicted in FIG. 21, it is contemplated that the inventive method and system (2100) collects oil by vapor (2102), which includes at least one phytochemical, making contact with wall (2105). Oil (2104) may then accumulate on wall (2105), where it then flows to an oil collector (not shown). Wall (2105) may be thermally coupled to cooling element (2106) to induce quick condensation of vapor (2102) into oil (2104). Cooling element (2106) may be a thermoelectric cooler or Peltier element. Wall (2105) may be thermally coupled to heating element (2108). Heating element (2108) may raise the temperature of oil (2104) for increasing viscosity or flow rate of oil (2104) for feeding an oil collector (not shown) connected to shoot (2107). The vacuum continues through shoot (2109).

As depicted in FIG. 22, it is contemplated that the inventive method and system (2200) includes a variation where wall (2202) defines a boundary between shoot (2107) and shoot (2109).

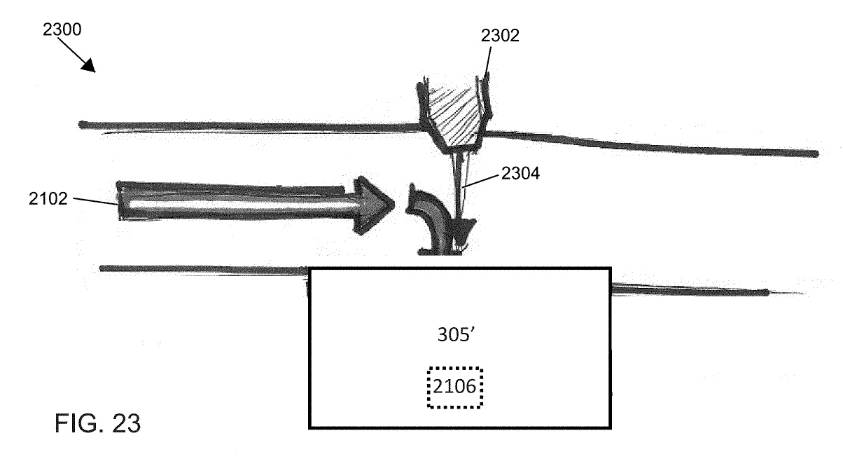

As depicted in FIG. 23, it is contemplated that the inventive method and system (2300) utilizes nozzle (2302) that emits gaseous stream (2304) for collecting at least one phytochemical in vapor (2102). Stream (2304) directs vapor (2102) towards collection chamber (305′), which may include cooling element (2106) for condensing vapor (2102) into an oil.

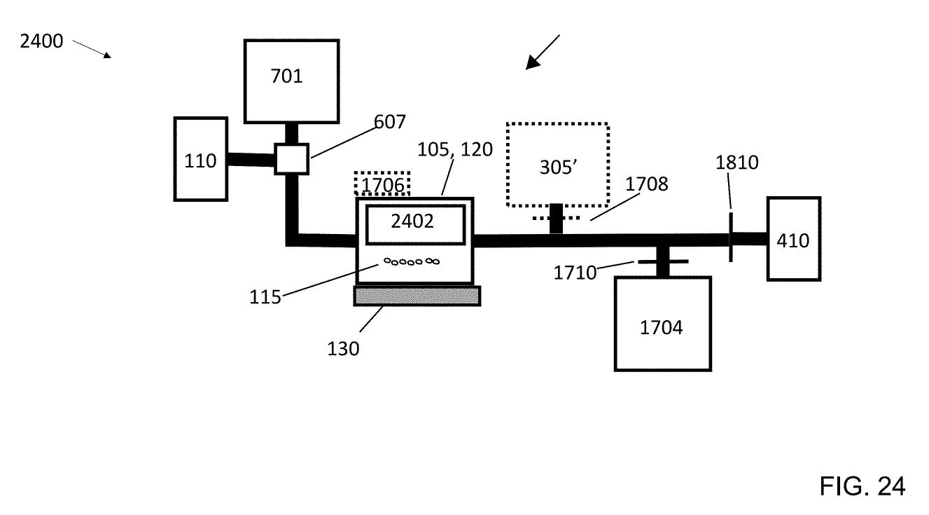

As depicted in FIG. 24, it is contemplated that the inventive method and system (2400) includes collector (2402) which at least partially extends into holder (105, 120). Collector (2402) may be operably coupled to (optional) downstream collection chamber (305′). In addition to or alternatively, collector (2402) may be removable from holder (105, 120) for collecting the phytochemical extract that accumulates on collector (2402).

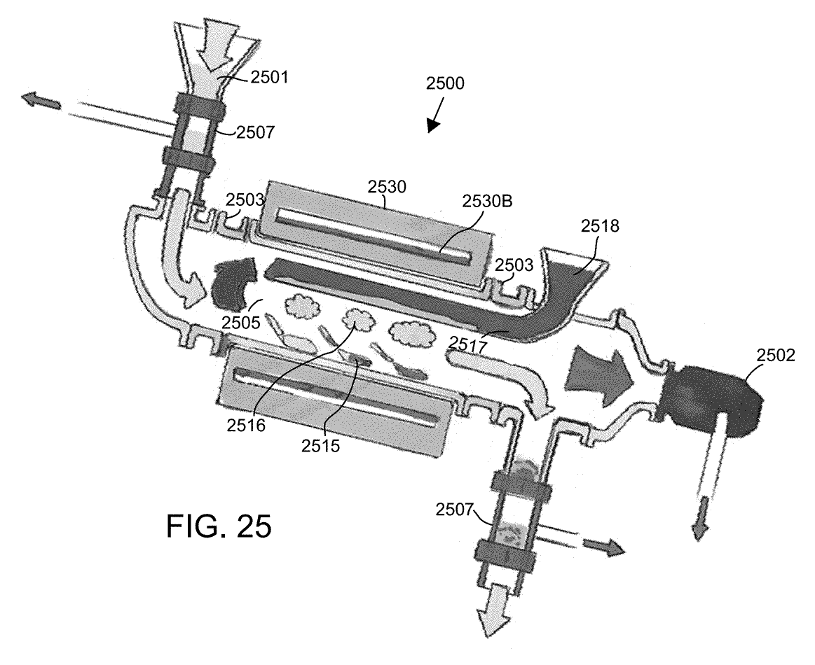

FIG. 25 shows a “cold finger” embodiment, including dispenser (2501), air locks (2507) where the arrows from the air locks (2507) point to fluid connections to vacuum pumps (not shown), rotary cuffs (2503) at each end of vacuum chamber (2505) which may be a clear quartz tube, heating source (2530) including IR lamp (2530B) surrounding the vacuum chamber (2505), “cold finger” (2517) in the vacuum chamber (2505) and supplied by LN2 feed (2518), vapor cloud (2516) and phytochemical composition (2515) tumbling in the rotating vacuum chamber (2505), and collection chamber (2502) where the arrow from the collection chamber (2502) points to fluid connections to a vacuum pump (not shown).

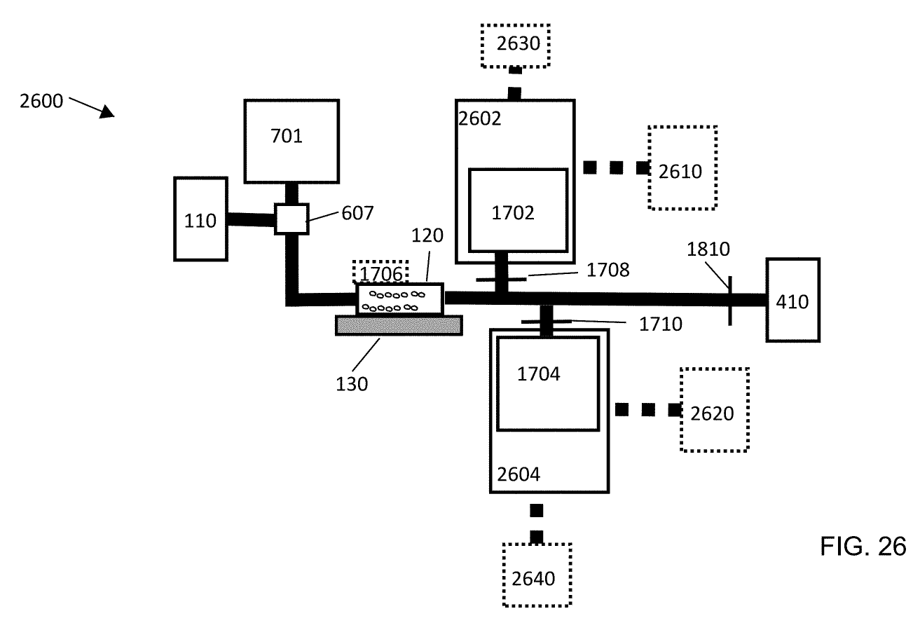

As depicted in FIG. 26, it is contemplated that the inventive method and system (2600) includes packagers (2602) and (2604) for respectively packaging the collected phytochemical extract and the processed material or composition. Packagers (2602) and (2604) may each be operably coupled to extraction pumps (2610) and (2620), which may establish or maintain a vacuum. This would ensure that either the collected phytochemical extract and/or the processed material or composition is under vacuum throughout the extraction and packaging process and not exposed to, for example, atmospheric air.

In addition or alternatively, packagers (2602) and (2604) may be respectively operably connected to refrigeration units (2630) and (2640), which may establish and maintain a temperature within packagers (2602) and (2604). This would ensure that neither the collected phytochemical extract nor the processed material or composition is exposed to ambient temperatures of system (2600).

Packagers (2602) and (2604) are adapted to place the collected extract or processed material/extract in one or more containers, include vials, vape pen cartridges, air-tight chambers, among other possible containers. If the first container is not air-tight, packagers (2602) and (2604) may package the first container in a second, air-tight package.

Packagers (2602) and (2604) may further process, before fully packaging, the collected phytochemical extract or the processed material or composition into another product or material to produce a topical composition, tincture, mixture, edible, eye drops, suppository (anal or vaginal), pill, among other possible products.

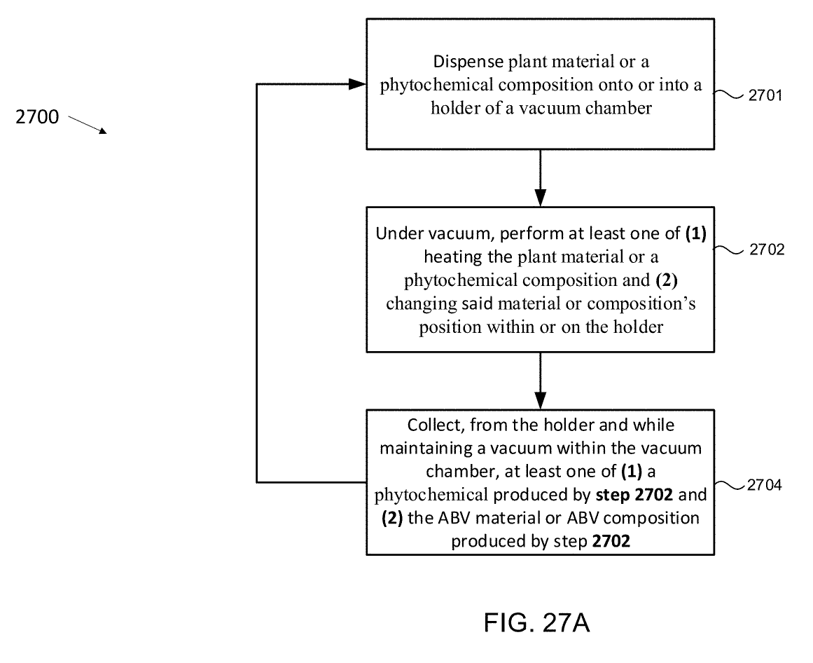

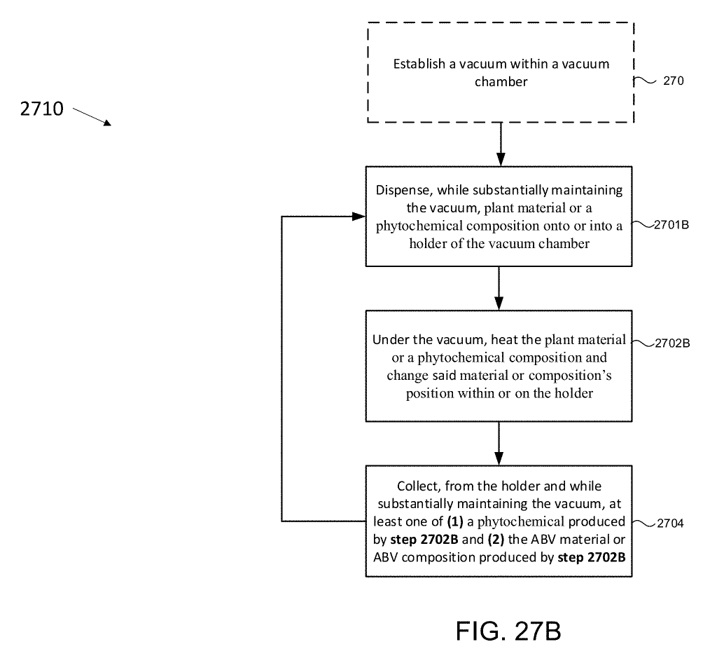

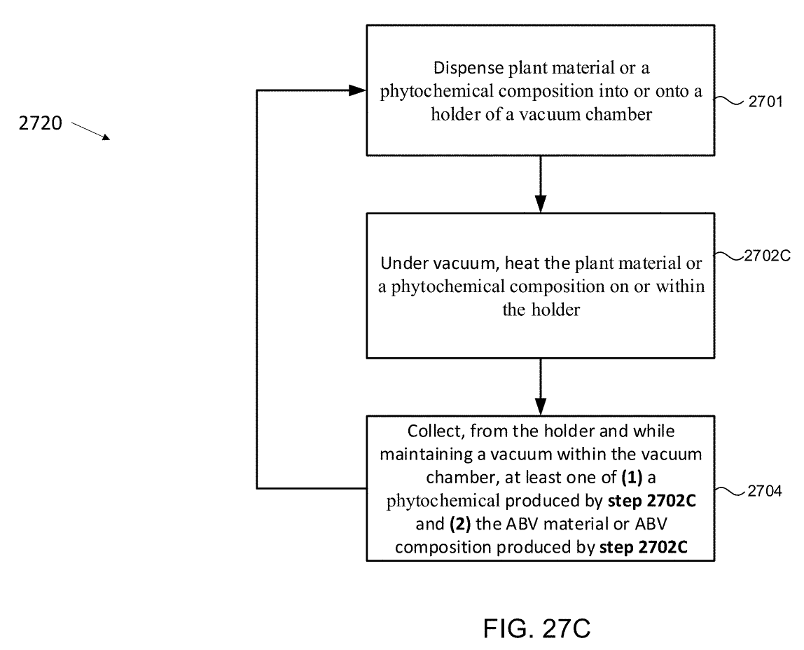

FIGS. 27A-C show embodiment method steps the inventive methods (2700), (2710), and (2720). Method (2700) may begin with step (2701) by dispensing plant material or a phytochemical composition onto or into a holder of a vacuum chamber. Step (2702) then, under vacuum, performs at least one of (1) heating the plant material or a phytochemical composition and (2) changing said material or composition's position within or on the holder. Step (2704) then collects, from the holder and while maintaining a vacuum within the vacuum chamber, at least one of (1) a phytochemical produced by step (2702) and (2) the ABV material or ABV composition produced by step (2702). Method (2700) may repeat back to step (2701).

Method (2710) may begin with step (270) by establishing a vacuum within a vacuum chamber. Then, step (2701B) dispenses, while substantially maintaining the vacuum, plant material or a phytochemical composition onto or into a holder of the vacuum chamber. Then, step (2702B) heats the plant material or a phytochemical composition and change said material or composition's position within or on the holder under the vacuum. Then, step (2704) collects, from the holder and while substantially maintaining the vacuum, at least one of (1) a phytochemical produced by step (2702B) and (2) the ABV material or ABV composition produced by step (2702B). Method (2710) may then repeat back to step (2701B).

Method (2720) may begin with step (2701), which dispenses plant material or a phytochemical composition into or onto a holder of a vacuum chamber. Then, step (2702C) heats the plant material or a phytochemical composition on or within the holder under a vacuum. Then, step (2704) collects, from the holder and while substantially maintaining the vacuum, at least one of (1) a phytochemical produced by step (2702C) and (2) the ABV material or ABV composition produced by step (2702C). Method (2720) may then repeat back to step (2701).

Referring to the figures generally, one theory of operational embodiment may be as follows. With the evacuation pump (110) disabled and the valve (205) open and located in the system (100) between the vacuum chamber (105) and the evacuation pump (110) as depicted, the plant material or a phytochemical composition (115) is placed in the vacuum chamber (105). It is contemplated that the vacuum chamber (105) is airtight and capable of maintaining a vacuum created and drawn therein by activation of the evacuation pump (110). It is also contemplated that the vacuum chamber (105) includes an opening and closing sealable door, port, or chamber to facilitate introduction and removal of the plant material or phytochemical composition (115) in and from the vacuum chamber (105).

Once the plant material or phytochemical composition (115) is placed inside the vacuum chamber (105), the valve (205) is adjusted to enable the evacuation of the vacuum chamber (105) when the evacuation pump (110) is activated thus creating at least a partial vacuum in the vacuum chamber (105). As the drawn vacuum increases, at least one phytochemical volatizes from and/or precipitates out of plant material or phytochemical composition (115) depending upon the dew-point temperature within the collector. If the temperature of the collector is below the dew point for and in accordance with the amount of vacuum in the vacuum chamber (110), the at least one phytochemical will volatize and collect (i.e. cold condense) in or on a collector. If the temperature of the collector is above the dew point for and in accordance with the amount of vacuum in the vacuum chamber (110), the at least one phytochemical precipitates out of the plant material or phytochemical composition and collects on a surface of a collector.

It is contemplated that the plant material (115) is from and belongs to the plant family Cannabaceae.

It is contemplated that the phytochemical composition (115) includes at least cannabinoids, terpenes, and combinations thereof.

It is contemplated that the heat source (130) comprises combustion of a fuel.

It is contemplated that the heat source (130) comprises an electrical heat element.

It is contemplated that the heat source (130) comprises a heated gas.

It is contemplated that the at least one phytochemical extracted and/or collected includes a cannabinoid, a terpene, or a combination thereof.

It is contemplated that in the case of phytochemical volatilization, a filter or trap (415) be placed between the source of volatilization and the point of cold condensation for ease of phytochemical collection, for increased system and production efficiency, and for improved system cleaning and maintenance.

In certain embodiments, the filer or trap (415) is located within the collection chamber (305, 305′).

It is contemplated that the inventive method and systems may include at least one processor, memory, software program, configurable hardware device, temperature sensor, pressure and/or vacuum sensor, valve control solenoid, temperature control solenoid, and/or other electromechanical system or device (none shown) to provide digital command and control of the inventive method and system.

It is contemplated that the inventive method and systems may include at least one processor, memory, software program, and configurable hardware device in wired or wireless communication with at least one temperature sensor, pressure and/or vacuum sensor, valve control solenoid, temperature control solenoid, and/or other electromechanical system or device (none shown) to provide remote digital command and control of the inventive method and system.

It is contemplated that the inventive method and systems may include at least one processor, memory, software program, and configurable hardware device in wired or wireless communication with at least one temperature sensor, pressure and/or vacuum sensor, valve control solenoid, temperature control solenoid, and/or other electromechanical system or device (none shown) to provide remote digital command and control of the inventive method and system via an intranet, Internet, or other communication network.

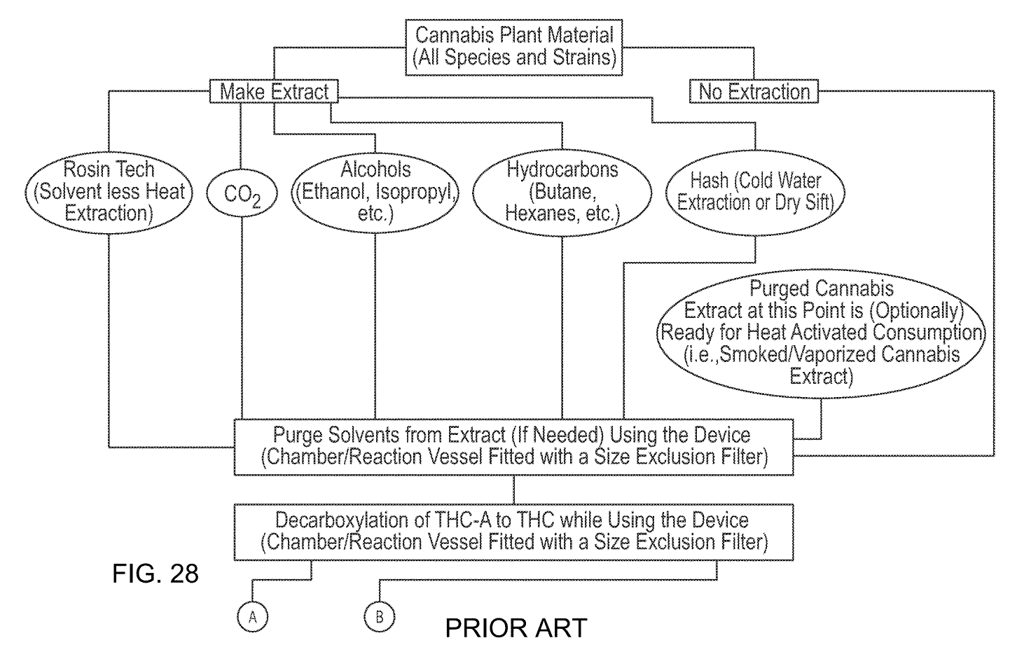

Most processes are made for other purposes other than Cannabis and Hemp. They were designed for the perfume industry and pharmaceutical industry—not specifically for Hemp and Cannabis. For example, FIG. 28 is a depiction of prior-art processes and is a copy of FIG. 1 of US 2017/0333505 A1.

(1) Current Extraction processes using solvents are CO2, Hydrocarbons, Alcohols—these require a 4 step process to (1) introduce the solvent/chemical to the plant material (2) extract oil, (3) purge solvents from the extract, (4) Decarboxylate THCa into THC.

CO2 has a 3 step process—(1) collection of terpenes in separate cylinder (2) a separate cylinder for cannabinoids and (3) separate cylinder for fats & lipids—this requires the reconstruction of the extract oil that has been broken apart into segments to be put back together again for use in a vape pen or other products. This causes a loss of the original structure of the compounds of the plant material—loss of terpenes and flavonoids.

Hydrocarbons—even after a good purge, there are small trace amounts of alcohol and hexane in the extract oil. This is harmful for the user's health.

(2) Current Solvent-less extraction processes include
(a) Cold Water Extraction (Bubble Hash)—(1) cold water is added as a solvent to extract the trichomes from the plant material and (2) dried until water moisture is removed completely; (3) cured; (4) heat activated to turn THCa into THC.
(b) Rosin (Heat)—(1) dry sift of plant material and (2) heat press dry sift or plant material; (3) add hemp seed oil to thin the viscosity for use in vape pens.

The solvent-less process of the present invention is: a one step process that decarboxylates the cannabis material and preserves the original terpenes into an extract/oil that has a viscosity that can go directly into a vape pen product, edibles, tincture and capsules. There is no need for additional steps. There are no harmful chemicals or solvents introduced to the plant material throughout any portion of the process. It is a 100% pure, clean extraction system made specifically for Cannabis and Hemp.

After a review of the oil produced by the present invention, which never has any solvents introduced to the plant material—there is zero residual in the final extract product—this is a breakthrough for the safety and health of the user/patient. The process of the invention allows the “entourage effect” wherein the medically viable compounds found in cannabis such as but not limited to cannabinoids and terpenes, interact in the mammalian endocannabinoid system, produces greater medical efficacy and safety. In addition, it increases the bio-availability and subsequent absorption of these medically viable compounds in the mammalian respiratory and gastrointestinal tracts.

### Example 1

Cannabis extraction process was performed using the following Extraction Process 1.

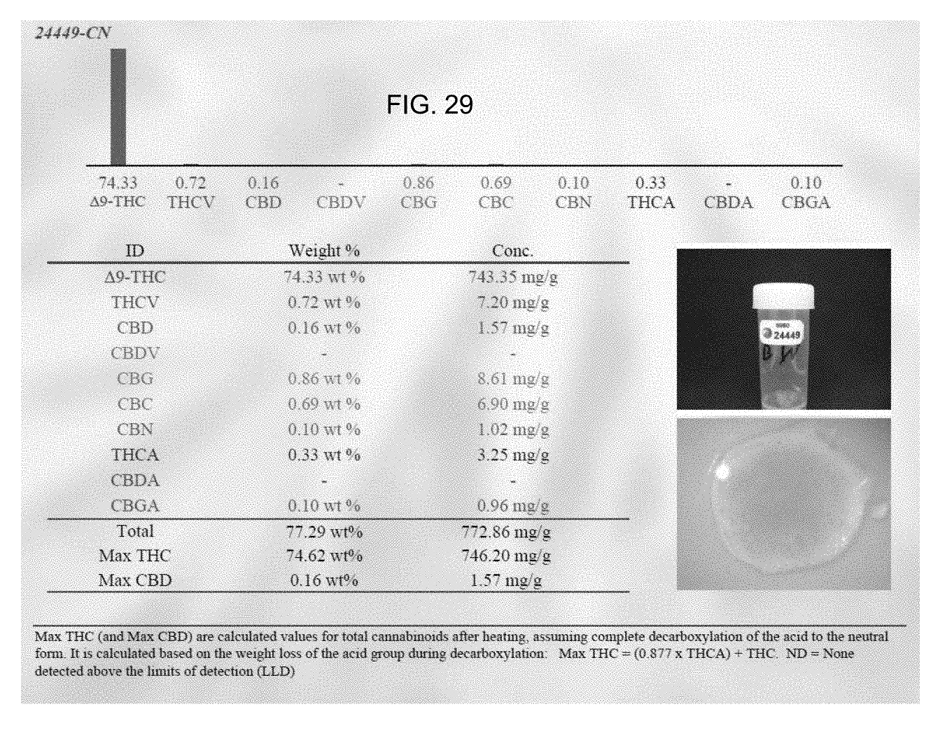

Process 1 started with 12 grams of cannabis plant material of strain, Black Water (California Cannabis Amendment 64 Recreational Cannabis). This cannabis extract was made using a solventless extraction process of cannabis plant material. This cannabis plant material was ground up for preparation of the extraction process. The cannabis material was placed onto the heating element in the extraction device. The vacuum chamber was lowered over the four cold-chilled glass collection chambers. Then the vacuum was pumped down to 10−5 torr. The chiller was turned on between 4 and 10 degrees Fahrenheit. The vaporizer was heated for a duration of 10 minutes reaching 280 degrees Fahrenheit. The cannabis material vaporized and collected on the four cold chilled collection chambers. The vacuum was released and the device chamber returned to atmospheric pressure and room temperature. The oil was collected manually and placed into a glass vile. The total oil extracted was 1.2 grams. Results are reported in FIG. 29.

Additional details regarding the invention are referred to in the attached claims of the application.

Having thus described several embodiments for practicing the inventive method, its advantages and objectives can be easily understood. Variations from the description above may and can be made by one skilled in the art without departing from the scope of the invention.

Accordingly, this invention is not to be limited by the embodiments as described, which are given by way of example only and not by way of limitation.

What is claimed is:

1. A method of and for extracting a phytochemical from plant material or a phytochemical composition, the method comprising the steps of:
dispensing the plant material or the phytochemical composition onto or into a holder that is within or at least partially defining a vacuum chamber;
substantially maintaining a first vacuum within the vacuum chamber;
under the first vacuum, sufficiently heating the plant material or the phytochemical composition to cause at least one phytochemical to volatize and/or precipitate from the plant material or phytochemical composition; and
collecting while substantially maintaining the first vacuum within the vacuum chamber, at least one of
(a) the at least one phytochemical, thereby creating a phytochemical extract and
(b) the processed plant material or processed phytochemical composition from the holder.

2. The method of claim 1, further comprising changing the position of the plant material or phytochemical composition within or on the holder.

3. The method of claim 2, with the changing step comprising changing the position of the plant material or phytochemical composition within or on the holder while performing the sufficiently heating step.

4. The method of claim 2, with the changing step comprising stirring the plant material or phytochemical composition within or on the holder.

5. The method of claim 2, with the changing step comprising at least one of vibrating the holder and rotating the holder.

6. The method of claim 1, with the collecting step comprising collecting Already Been Vaped (ABV) plant material or ABV phytochemical from the holder.

7. The method of claim 1, further comprising packaging, into a container and under the first vacuum or a second vacuum, at least one of
(a) the collected phytochemical extract, and
(b) the processed plant material or the processed phytochemical composition collected from the holder.

8. The method of claim 1, packaging into a container at least one of
(a) the collected phytochemical extract, and
(b) the processed plant material or the processed phytochemical composition collected from the holder.

9. The method of claim 1, further comprising,
before dispensing the plant material or the phytochemical composition onto or into the holder:
dispensing the plant material or the phytochemical composition in a first holding chamber;
establishing or increasing a second vacuum of the first holding chamber that is the same or a substantially similar vacuum quality to the first vacuum;
fluidly coupling the first holding chamber with the vacuum chamber.

10. The method of claim 1, with the collecting step comprising:
dispensing the processed plant material or the phytochemical composition in a second holding chamber that is both in fluid communication with the vacuum chamber and under the first vacuum;
isolating the second chamber from the vacuum chamber, thereby fluidly decoupling the second chamber from the vacuum chamber, and
evacuating the processed plant material or the phytochemical composition from the second holding chamber.

11. A phytochemical extraction system comprising:
a vacuum chamber arranged to maintain a vacuum;
a holder arranged to hold a plant material or a phytochemical composition, the holder arranged within the vacuum chamber or at least partially defining the vacuum chamber, the holder defining a first aperture arranged for evacuating the plant material or the phytochemical composition from the holder;
an evacuation pump in fluid communication with the vacuum chamber and arranged to create a vacuum within the vacuum chamber;
a collector arranged to collect at least one phytochemical that volatizes and/or precipitates from the plant material or the phytochemical composition; and
a heat source arranged to increase the temperature of the plant material or phytochemical composition in or on the holder when the heat source is operating,
the vacuum chamber and the evacuation pump operably coupled for establishing an amount of vacuum that is sufficient to cause, in combination with the increase of the temperature caused by the heat source, the at least one phytochemical to volatize and/or precipitate from the plant material or the phytochemical composition, thereby processing the plant material or the phytochemical composition and creating a phytochemical extract.

12. The system of claim 11, with the first aperture being further arranged for receiving the plant material or the phytochemical composition into or onto the holder.

13. The system of claim 11, with the holder further defining a second aperture arranged for receiving the plant material or the phytochemical composition into or onto the holder.

14. The system of claim 11, further comprising at least one vibration element operably coupled to the holder and arranged to change the position of the plant material or phytochemical composition within or on the holder when the at least one vibration element vibrates.

15. The system of claim 11, further comprising at least one rotatable connection operably coupled to the holder and arranged to change the position of the plant material or phytochemical composition within or on the holder when the at least one rotatable connection rotates.

16. The system of claim 11, further comprising at least one stirrer arranged to change the position of the plant material or phytochemical composition within or on the holder when the at least one stirrer operates.

17. The system of claim 11 further comprising a dispenser arranged to dispense, upstream of the holder, the plant material or the phytochemical composition.

18. The system of claim 17, with the dispenser further arranged within the vacuum chamber.

19. The system of claim 11 further comprising at least one of a first holding chamber and a second holding chamber, the first holding chamber and the second holding chamber adapted to isolate the respective chamber from the vacuum chamber and establish fluid communication with the respective chamber and the vacuum chamber, the first holding chamber arranged to dispense the plant material or the phytochemical composition into the vacuum chamber or onto or into the holder and the second holding chamber arranged to receive the processed plant material or the processed phytochemical composition.

20. The system of claim 11, where the collector is arranged at least partially within the holder.

21. The system of claim 11, where the holder defines a cylindrical shape and at least partially defining the vacuum chamber.

22. The system of claim 11, with the heat source comprises at least one infrared heating element.

23. The system of claim 11, with the holder comprising a material at least substantially transparent to at least one wavelength emitted by the heat source.

24. The system of claim 11, with the heat source arranged or arrangeable to surround at least a portion of the holder.
